(19) United States
(12) Patent Application Publication
(10) Pub. No.: US 2017/0266264 A1
(43) Pub. Date: Sep. 21, 2017

(54) TRUNCATED LYSOSOMAL ACID LIPASE

(71) Applicant: ALEXION PHARMA LLC, New Haven, CT (US)

(72) Inventors: Erin Renae Treece, Dunstable, MA (US); Nelson Hsia, Cambridge, MA (US); Zhinan Xia, Wellesley, MA (US); Anthony Quinn, Chestnut Hill, MA (US)

(21) Appl. No.: 15/386,595

(22) Filed: Dec. 21, 2016

Related U.S. Application Data
(63) Continuation of application No. 14/377,990, filed on Aug. 11, 2014, now abandoned.
(60) Provisional application No. 61/605,850, filed on Mar. 2, 2012.

(51) Int. Cl. A61K 38/46; C12N 9/20

(52) U.S. Cl. CPC A61K 38/465; C12N 9/20; C12Y 301/01013

(57) ABSTRACT

Recombinant human lysosomal acid lipase (rhLAL) containing an N-terminal truncation, a composition of truncated recombinant human LAL (TLAL), an isolated mixture comprising TLAL and at least one other form of rhLAL are disclosed. A method of purifying TLAL from a mixture of LAL proteins, pharmaceutical compositions comprising TLAL and methods of producing TLAL are further disclosed.

### RELATED APPLICATIONS

This application is a continuation of Ser. No. 14/377,990, filed Aug. 11, 2014, which is the U.S. National Stage of International Application No. PCT/US2013/028688, filed Mar. 1, 2013, published in English, and claims the benefit of U.S. Provisional Application No. 61/605,850, filed on Mar. 2, 2012. The entire teachings of the foregoing applications are incorporated herein by reference.

### SEQUENCE LISTING

The instant application contains a Sequence Listing which has been submitted electronically in ASCII format and is hereby incorporated by reference in its entirety. Said ASCII copy, created on Nov. 15, 2016, is named “085US_SEQ_ST25.TXT” and is 179 KB in size.

### BACKGROUND OF THE INVENTION

Lysosomal acid lipase (LAL) hydrolyzes cholesteryl esters or triglycerides that are internalized into the lysosome via low density lipoprotein (LDL) particles receptor-mediated endocytosis. Defects in LAL lead to a condition known as LAL deficiency characterized by the massive accumulation of cholesteryl esters (CE) and triglycerides (TG) in vital tissues of affected individuals. Wolman disease, a severe form of the LAL deficiency, typically leads mortality within one year of age due to complications associated with the massive accumulation of lipids in vital organs. Cholesteryl ester storage disease (CESD), a late onset LAL deficiency, can be identified in various stages of life with varying degrees of severity.

Human LAL (hLAL, EC 3.1.1.13) is encoded by the gene known as LIPA (NCBI Accession no. U08464.1) which contains nine coding exons localized in chromosome 10. The unprocessed full-length human LAL with its native signal peptide contains 399 amino acid residues. Allelic variations of human LIPA have been characterized and missense as well as nonsense and deletion mutations linked to WD and CESD have been identified (see, e.g., Lugowska et al., Lysosomal Acid Lipase Deficiency: Wolman Disease and Cholesteryl Ester Storage Disease, Lysosomal Storage Diseases (2012) Vol. 10:1-8).

Molecular and biochemical analyses of the LAL protein have suggested that two forms of human LAL may exist in human liver. A shorter form (41 kDa) of naturally occurring human LAL was detected along with a full-length hLAL (56 kDa) in human liver extracts (Ameis, D. et al., Eur. J. Biochem. 219:905-914, 1994). However, subsequent attempts to express and isolate the short form of LAL in vitro have consistently failed due to unknown biological mechanisms associated with post-translational modification of LAL (see, Zschenker et al. (2004)). Zschenker et al. (2004) concluded that the N-terminal region of LAL is essential for protein folding, stability, secretion and enzyme activity.

Enzyme replacement therapy for the treatment of LAL deficiency is currently being investigated as a viable treatment. Therefore, a need exists to provide various active forms of recombinant human LAL that could potentially minimize dosing amount and frequency and enhance the quality of life of patients with LAL deficiency.

### SUMMARY OF THE INVENTION

The present invention is based on the discovery that while the N-terminal region of rhLAL is needed for proper expression and processing of the protein, it is not required for enzyme activity. A full-length rhLAL appears to undergo a post-translational modification (i.e., proteolytic cleavage) resulting in production of truncated hLAL protein missing the N-terminal portion. It is unexpected and surprising to discover that N-terminal truncated form of rhLAL (hereinafter, “TLAL”) having 323-371 amino acid residues of C-terminal portion exhibit equivalent or higher enzyme activity as compared to the full-length protein.

When recombinant human lysosomal acid lipase (rhLAL) is produced in mammalian cells, the cell culture medium contains not only LAL protein of the expected size (apparent molecular weight of 56 kDa by SDS polyacrylamide gel electrophoresis), but also a lower molecular weight (truncated) form (apparent molecular weight of 41 kDa on SDS-PAGE). Subsequent analyses revealed that this truncated rhLAL contains 324 amino acid residues of C-terminal portion of LAL and occurs by proteolysis of the 56 kDa LAL protein. TLALs have an N-terminal truncation up to 50, 60 or 70 amino acid residues.

Direct expression of a 41 kDA TLAL in mammalian cell line did not produce the protein above detectable ranges, suggesting the nascent LAL protein missing the N-terminal portion is rapidly degraded in the endoplasmic reticulum (ER), whereas inclusion of an N-terminal sequence enables the rhLAL to enter into the secretory pathway. However, production of the truncated form of rhLAL (TLAL) by proteolytic cleavage of the full-length rhLAL led to the discovery that the 41 kDa recombinant TLAL protein retained LAL activity, that is at least 50% or even higher than of that observed in the full-length 56 kDa rhLAL.

Accordingly, TLAL described herein is a recombinant protein that contains an N-terminal truncation from full-length LAL and retains enzymatic activity. In some aspects, TLAL exhibits enzyme activity at least 50%, 60%, 70%, 80%, 90% or 100% of the activity observed in a full-length rhLAL. In some aspects, TLAL has enzyme activity levels equivalent to or higher than that of a full-length LAL. The present invention provides a composition comprising an isolated TLAL, a mixture of the isolated rhLAL comprising a TLAL and at least one other form of rhLAL (e.g., full-length rhLAL or another TLAL). The present invention also provides a method of purifying TLAL from an isolated mixture comprising TLAL and at least one other form of rhLAL. The present invention also includes methods of purifying away a TLAL from an isolated mixture of rhLAL proteins. The present invention provides a pharmaceutical composition comprising TLAL and pharmaceutically acceptable carrier or excipient. Also included are methods of increasing LAL activity by N-terminal truncation of a full-length rhLAL and methods of producing a truncated rhLAL in mammalian cell culture.

In one aspect, the invention provides a composition comprising an isolated recombinant truncated lysosomal acid lipase (TLAL). In one embodiment, the TLAL has at least 90%, 95% or 99% identity to any one of the amino acid sequences set forth in SEQ ID NOs:10-58. In another embodiment, the TLAL is selected from the group consisting of SEQ ID NOs: 10-58. In one embodiment, the TLAL comprises the amino acid sequence set forth in SEQ ID NO:10. In one embodiment, the TLAL comprises the amino acid sequences set forth in SEQ ID NO:11. In one embodiment, the TLAL has an enzyme activity that is at least 50%, 60%, 70%, 80%, 90%, 100%, 150%, or 200% of that of a full-length recombinant human LAL (rhLAL). In another embodiment, the TLAL has an enzyme activity that is at least equal to or higher than that of the full-length rhLAL.

In another aspect, the invention provides a composition comprising an isolated recombinant TLAL, wherein the TLAL comprises the amino acid sequence set forth in SEQ ID NO: 10. In one embodiment, the composition further comprises mammalian culture medium.

In another aspect, the invention provides a composition comprising an isolated recombinant TLAL, wherein the TLAL comprises the amino acid sequence set forth in SEQ ID NO: 11.

In another aspect, the invention provides a composition comprising an isolated mixture of rhLAL, the mixture comprising at least one form of recombinant TLAL. In one embodiment, the mixture further comprises a full-length rhLAL. In another embodiment, the recombinant TLAL comprises the amino acid sequence set forth in SEQ ID NO: 10. In yet another embodiment, the recombinant TLAL comprises the amino acid sequence set forth in SEQ ID NO: 11.

In another aspect, the invention provides a composition comprising an isolated recombinant human LAL, wherein the LAL is a fusion protein comprising a second moiety. In one embodiment, the second moiety is fused to the N-terminus of the LAL, the C-terminus of the LAL, or internally to any amino acid residue position between Pro31 and Gly77 of the LAL. In another embodiment, the second moiety is fused to the LAL recombinantly. In yet another embodiment, the second moiety is fused to rhLAL via a linker.

In another aspect, the invention provides a pharmaceutical composition comprising the composition of claim 1. In one embodiment, the pharmaceutical composition further comprises a buffer. In one embodiment, the pharmaceutical composition further comprises an excipient. In one embodiment, the pharmaceutical composition further comprises a salt.

In another aspect, the invention provides a method of purifying an rhLAL from an isolated mixture comprising a full-length rhLAL and a recombinant TLAL, the method comprising purifying away the recombinant full-length rhLAL or TLAL from the mixture. In one embodiment, the purification is performed using one or more methods selected from the group consisting of hydrophobic interaction chromatography, affinity chromatography, ion exchange chromatography, size-exclusion chromatography, selective precipitation, crystallization, and tangential flow filtration.

In another aspect, the invention provides a method for treating a LAL deficiency in a patient, the method comprising: administering to the patient a composition of the invention in an amount effective to treat a LAL deficiency. In one embodiment, the LAL deficiency is Wolman disease (WD). In another embodiment, the LAL deficiency is cholesteryl ester storage disease (CESD). In another embodiment, the TLAL or rhLAL is administered intravenously. In another embodiment, the TLAL or rhLAL is administered via a pump. In another embodiment, the amount is in the range of 0.1 to 20 mg of TLAL or rhLAL per kg of body weight.

### BRIEF DESCRIPTION OF THE FIGURES

FIGS. 1A-1J depict the amino acid sequences of unprocessed full-length LAL (SEQ ID NO:1) and various length TLAL proteins (SEQ ID NOs: 2-58).

FIG. 2 shows the SDS-PAGE analysis of rhLAL digestion by peptide N-glycosidase F-amidase (PNGase F). Lane 1, molecular weight markers; Lane 2, PNGase F only; Lane 3, rhLAL protein (loading concentration, 4 μg) produced in transgenic chicken as described in WO2011/133960; Lane 4, rhLAL (loading concentration, 4 μg) produced in transgenic chicken, digested with PNGase F; Lane 5, rhLAL (loading concentration, 8 μg) produced in transgenic chicken; Lane 6, rhLAL (loading concentration, 4 μg) produced in HEK 293 cells transfected with pTT22-LAL, undigested with PNGase F; Lane 7, rhLAL (loading concentration, 4 μg) produced in HEK 293 cells transfected with pTT22-LAL, digested with PNGase F; and Lane 8 rhLAL (loading concentration, 8 μg) produced in HEK 293 cells transfected with pTT22-LAL, undigested with PNGase F. “SGG . . . ” is the N-terminal amino acid sequence corresponding to positions 22-399 of SEQ ID NO:1. “KGP . . . ” is the N-terminal amino acid sequence of 76-399 of SEQ ID NO:1 (i.e., SEQ ID NO:11).

FIG. 3 depicts an image of chemiluminescence of a Western blot of rhLAL proteins treated with PNGase. Lane 1, PNGase F only; Lane 2, rhLAL (4 μg) produced in transgenic chicken, untreated; Lane 3, rhLAL (4 μg) produced in transgenic chicken, treated with PNGase F; Lane 4, rhLAL (4 μg) produced in HEK 293 cells transfected with pTT22-LAL, untreated; and Lane 5, rhLAL (4 μg) produced from HEK 293 cells transfected with pTT22-LAL, treated with PNGase F.

FIG. 4A depicts a diagram of transient expression and purification steps in HEK 293 cells. FIG. 4B depicts a S200 chromatogram of a size exclusion chromatography (SEC) for rhLAL produced in HEK 293 cells transfected with pTT22-LAL.

FIG. 5 demonstrates human macrophage (NR8383) cellular uptake as measured in subcellular LAL activity (units/cell) using various concentrations of rhLAL proteins (HEK produced LAL, left bars; chicken produced LAL, right bars) co-incubated.

FIG. 6 depicts the nucleic acid sequence of a linker containing both the enterokinase (ek) cleavage site (“DDDDK”; SEQ ID NO:64) and the FLAG affinity sequence.

FIG. 7 depicts the amino acid sequence (SEQ ID NO:60) of the FLAG affinity sequence and enterokinase (ek) cleavage site.

FIG. 8A depicts the amino acid sequence of an rhLAL (full-length, unprocessed LAL) containing the FLAG affinity and ek cleavage site (underlined) (SEQ ID NO:61). Bovine enterokinase cleaves C-terminal to DDDDK (SEQ ID NO:64), thereby producing TLAL-G77 protein of SEQ ID NO:10. FIG. 8B depicts the amino acid sequence of an rhLAL (full-length, unprocessed LAL) containing the FLAG affinity and ek cleavage site (underlined) (SEQ ID NO:68) designed to produce TLAL-K76 protein of SEQ ID NO:11 upon ek digestion.

FIG. 9 depicts the forward and reverse primers designed to introduce the ek recognition site into the LAL coding sequence (SEQ ID NOs: 62 and 63). The primers contain the FLAG affinity/ek cleavage sequence.

FIG. 10 illustrates a map of a vector constructed from pTT22 that contains the ekLAL coding sequence.

FIG. 11A is an image of an SDS-polyacrylamide gel stained with Coomassie blue. ekLAL (SEQ ID NO:61) was produced in HEK293 cells transfected with pTT22-EK-gpLAL and purified according the methods described in Example 2. Proteolytic digestion of ekLAL-HEK and subsequent purification of digestion products were performed as described in Example 3. Lane 1, molecular weight markers; Lane 2, 2 μg of α-FLAG column starting sample; Lane 3, 2 μg of α-FLAG flow through (“TLAL-HEK,” a proteolytic digestion product corresponding to TLAL-G77); Lane 4, 12 μL of α-FLAG wash; and Lane 5, 2 μg of α-FLAG fractions (“ekLAL-HEK,” an uncleaved product). FIG. 11B is an image of an SDS-polyacrylamide gel. MW: molecular marker; Lane 1, 0.5 μg of full-length rhLAL produced in transgenic chicken; Lane 2, 0.5 μg of full-length rhLAL produced in HEK293 cells; Lane 3, 0.5 μg of TLAL-K76 of SEQ ID NO:11 produced in HEK293 cells.

FIG. 12 is a bar graph depicting lipase activity levels of TLAL-G77 (SEQ ID NO:10) and ekLAL produced in HEK293 cells (SEQ ID NO:61). ekLAL-HEK protein containing was purified from conditioned medium and frozen for subsequent analyses. “Unaltered” samples were assayed immediately after thawing as positive control. “Undigested” samples were incubated at room temperature for 6 days without the presence of enterokinase. ekLAL was digested with enterokinase for 6 days at room temperature. The treated but uncleaved ekLAL (“digested ekLAL-HEK”) and the cleaved product (“digested TLAL-HEK” corresponding to TLAL-G77 of SEQ ID NO:10) were isolated and purified by affinity chromatography and subjected to enzymatic activity assay.

FIG. 13 is a graph depicting enzyme activity levels of recombinant LAL at various protein concentrations.

FIG. 14 depicts enzymatic activity levels of TLAL-K76 (SEQ ID NO:11) as compared to full-length rhLALs produced in transgenic avian (LAL-EW) and HEK293 (LAL-HEK) which were subject to similar ek treatments.

### DETAILED DESCRIPTION OF THE INVENTION

The present invention relates to a group of isolated truncated forms of recombinant human LAL (“TLAL”). Some TLAL proteins retain enzymatic activity and are capable of hydrolyzing cholesteryl esters (CE) and triglycerides (TG). The compositions described herein comprise one or more TLAL proteins or polypeptides. Proteins or polypeptides referred to herein as “isolated” are proteins or polypeptides that are purified to a state beyond that in which they exist in cells, and include proteins or polypeptides obtained by methods described herein, similar methods or other suitable methods, and also include essentially pure proteins or polypeptides, proteins or polypeptides produced by chemical synthesis or by combinations of biological and chemical methods, and recombinant proteins or polypeptides which are isolated. Further, proteins or polypeptides referred to herein as “recombinant” are produced by the expression of recombinant nucleic acids.

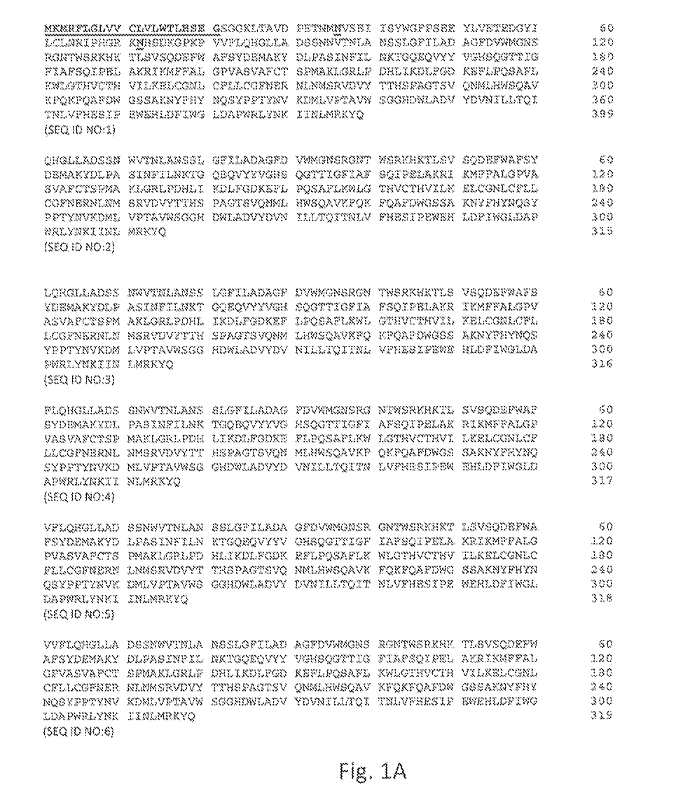

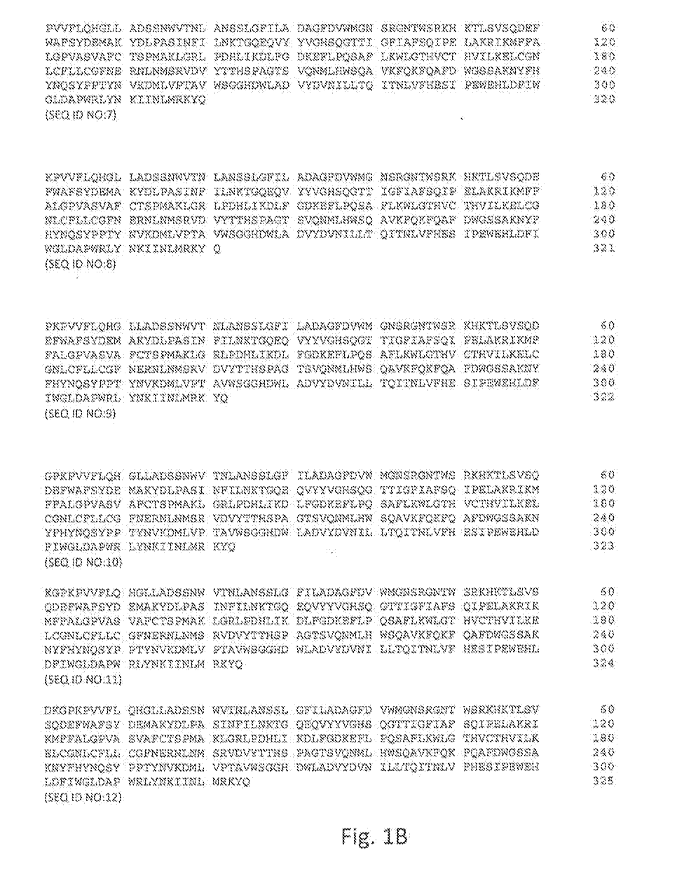

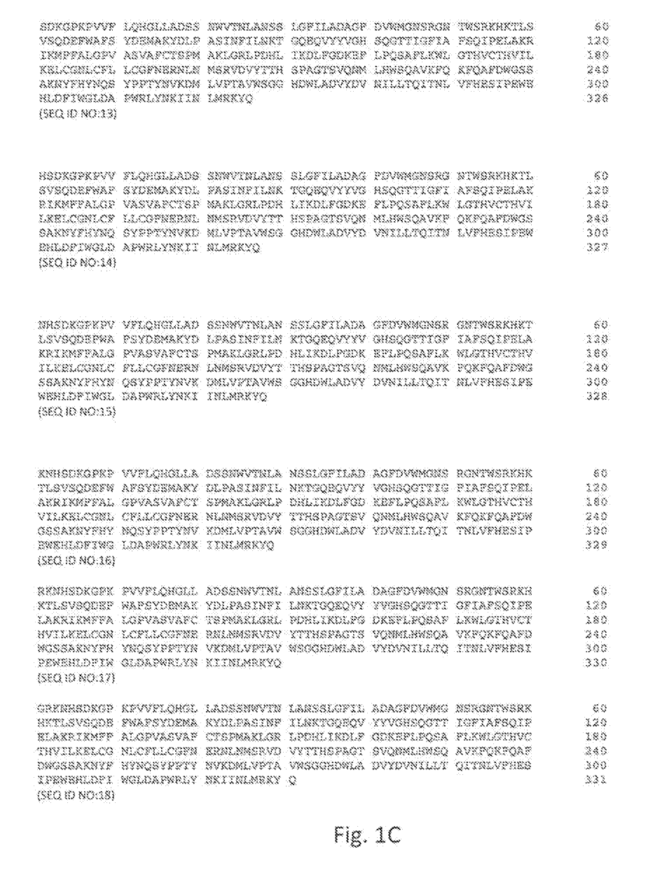

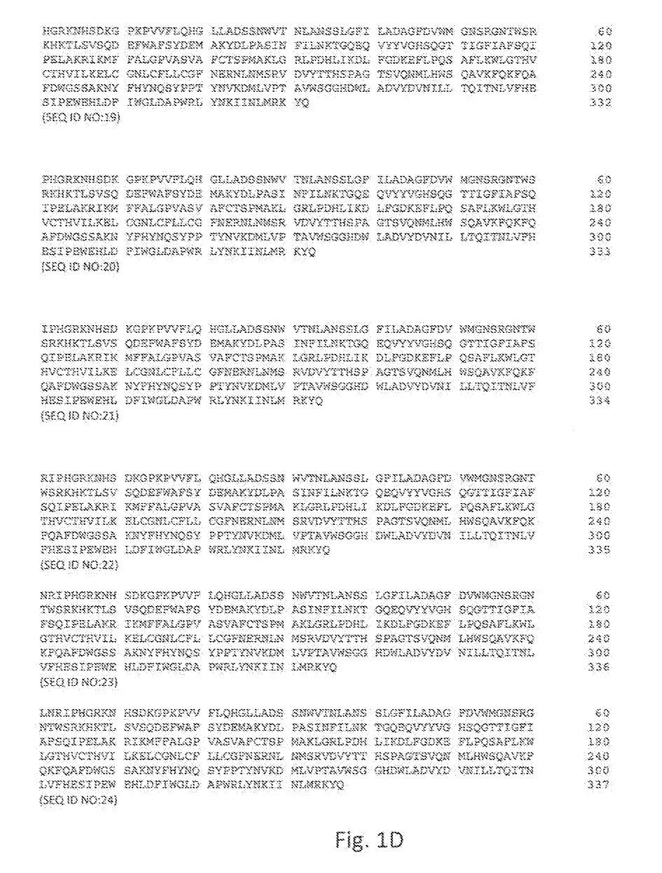

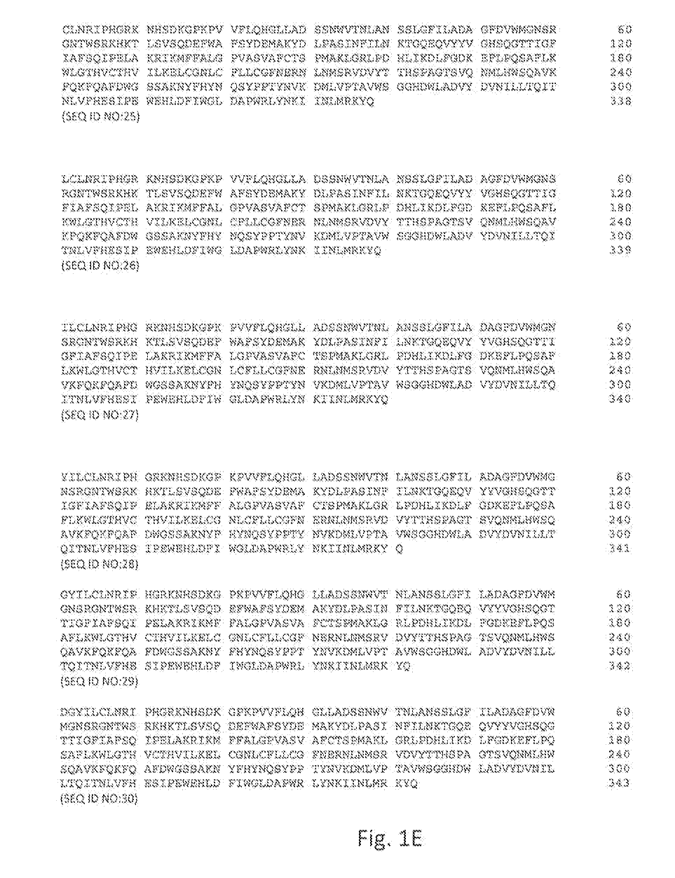

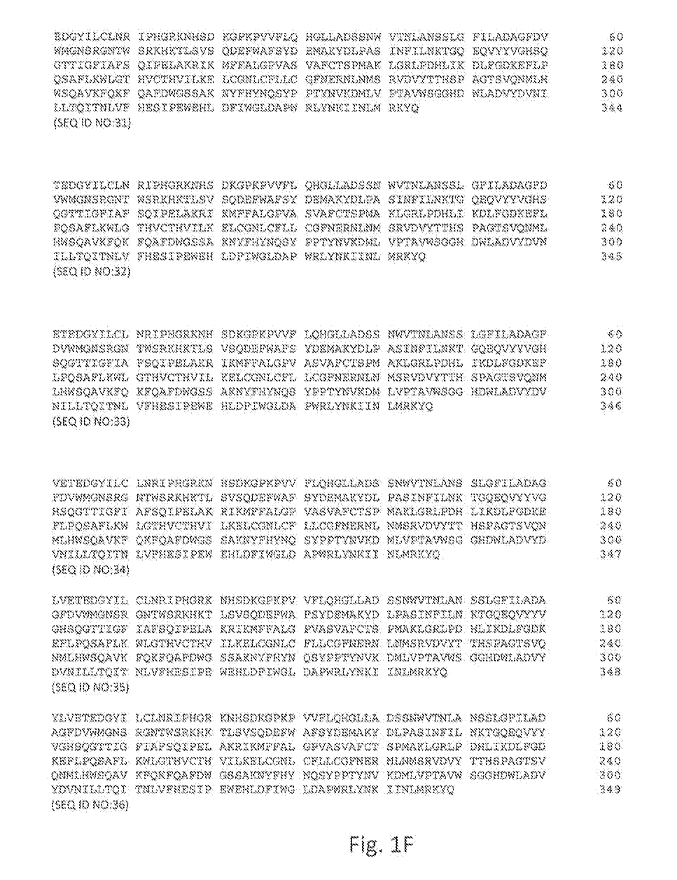

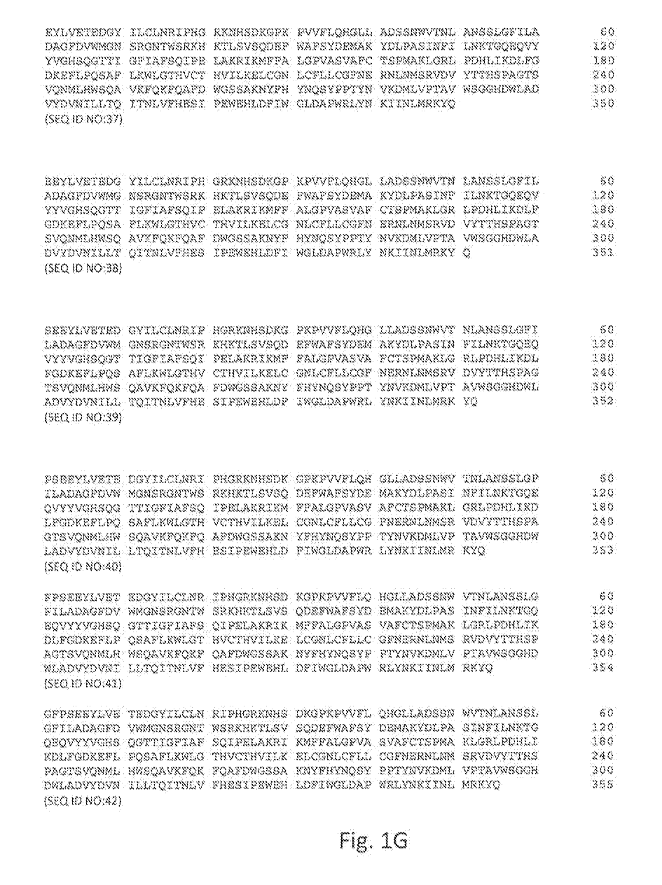

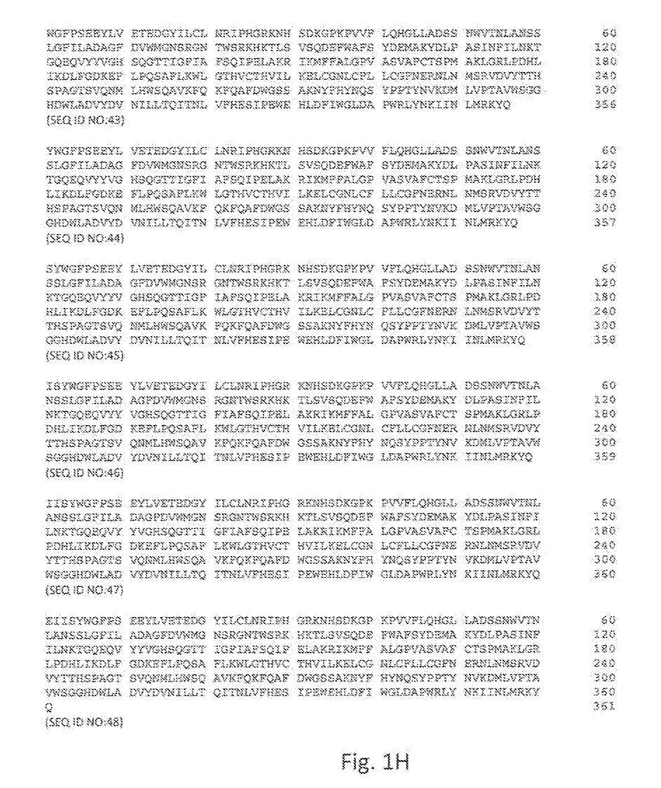

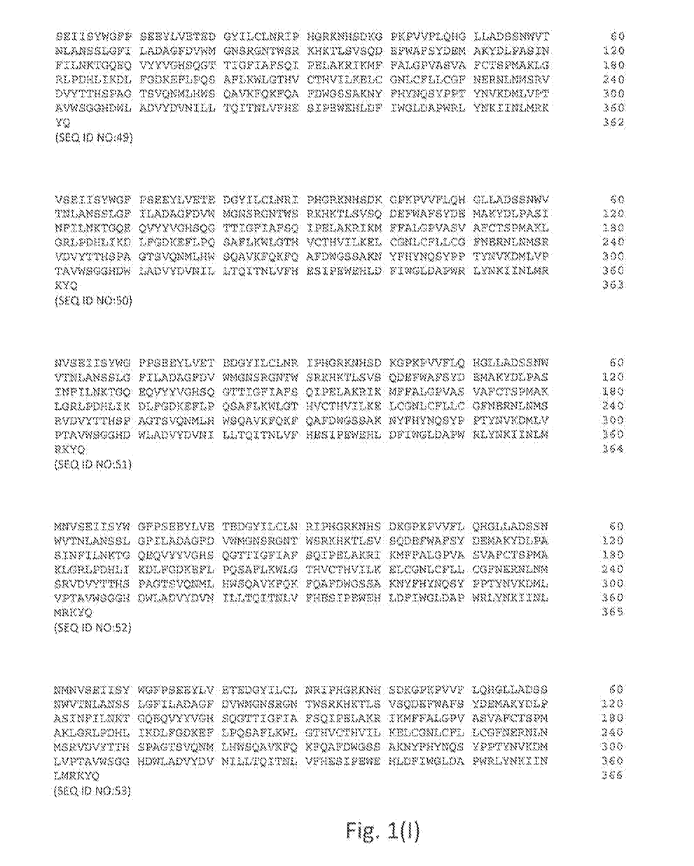

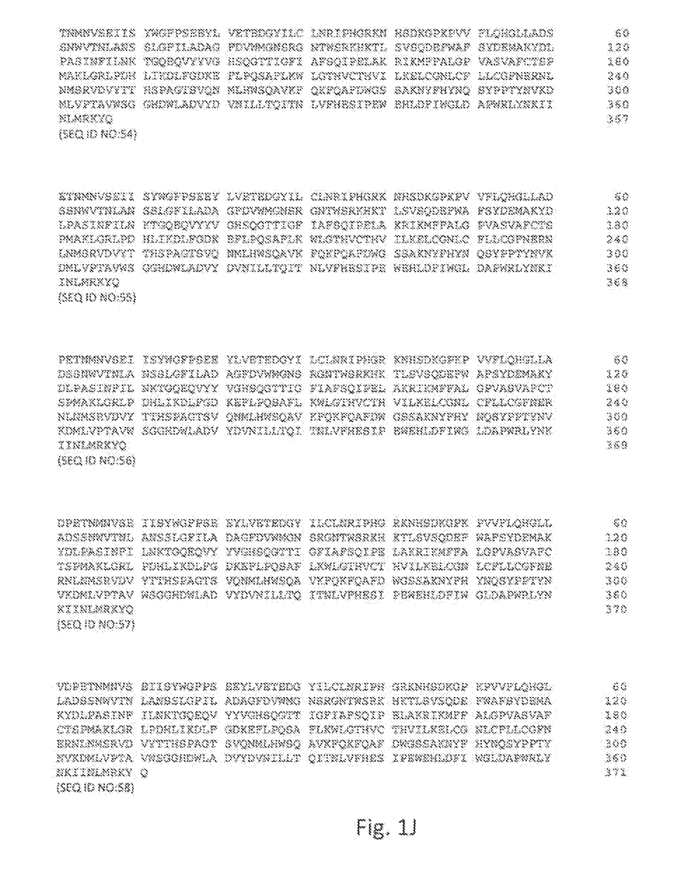

Positions of the amino acid residues of recombinant human LAL (rhLALs) are referred to as they are numbered from the N-terminal Met1 to the C-terminal Gln399 in unprocessed rhLAL. The corresponding amino acid residue positions from 1 through 399 are set forth in SEQ ID NO:1 (FIG. 1). It is to be understood that the signal peptide sequence described herein is any of the first 21 to 27 amino acid residues at the N-terminus of rhLAL having a native human signal peptide sequence (e.g., SEQ ID NO:1). Accordingly, a full-length unprocessed recombinant human lysosomal acid lipase (rhLAL) is 399 amino acid residues long (SEQ ID NO:1), including the native signal peptide sequence at the N-terminus as shown in FIG. 1. After cleavage of the signal peptide, a processed full-length rhLAL can be 372 to 378 amino acid residues long, depending on the length of the signal peptide sequence cleaved off from the 399 amino acid residues long rhLAL. Thus, the full-length human rhLAL without the signal peptide can include rhLAL having 378 amino acid residues (i.e., Ser22 through Gln399), 377 amino acid residues (i.e., Gly23 through Gln399), 376 amino acid residues (i.e., Gly24 through Gln399), 375 amino acid residues (i.e., Lys25 through Gln399), 374 amino acid residues (i.e., Leu26 through Gln399), 373 amino acid residues (i.e., Thr27 through Gln399), or 372 amino acid residues (i.e., Ala28 through Gln399). As used herein, the term “full-length rhLAL” includes both the unprocessed and processed rhLALs described above.

### TLAL Proteins

All truncated forms of recombinant LAL (“TLALs”) described herein comprise a portion of full-length human LAL (“hLAL”).

In one embodiment, the TLAL described herein can be any one of TLAL having 315 to 371 amino acid residues long as set forth in SEQ ID NOs:2-58. In a preferred embodiment, the TLAL can be 323 to 371 amino acid residues long, having the amino acid sequence of the C-terminal region of hLAL (SEQ ID NOs:10-58).

In one embodiment, a TLAL comprises the C-terminal 323 amino acid residues of hLAL, and in another embodiment, consists of those 323 amino acid residues. The N-terminus of this truncated form of rhLAL corresponds to Gly77 of hLAL. This truncated form is designated TLAL-G77 (SEQ ID NO:10).

In another embodiment, a TLAL comprises the C-terminal 324 amino acid residues of hLAL, and in another embodiment, consists of those 324 amino acid residues. The N-terminus of this truncated form of rhLAL corresponds to Lys76 of hLAL. This truncated form is designated TLAL-K76 (SEQ ID NO:11).

Another recombinant TLAL described herein comprises the C-terminal 325 amino acid residues of hLAL, and in another embodiment, consists of those 325 amino acid residues. The N-terminus of this truncated form of rhLAL corresponds to Asp75 of hLAL. This truncated form is designated TLAL-D75 (SEQ ID NO:12).

Another recombinant TLAL described herein comprises the C-terminal 315 amino acid residues of hLAL, and in another embodiment, consists of those 315 amino acid residues. The N-terminus of this truncated form of rhLAL corresponds to Gln85 of hLAL. This truncated form is designated TLAL-Q85 (SEQ ID NO:2).

Another recombinant TLAL described herein comprises the C-terminal 316 amino acid residues of hLAL, and in another embodiment, consists of those 316 amino acid residues. The N-terminus of this truncated form of rhLAL corresponds to Leu84 of hLAL. This truncated form is designated TLAL-L84 (SEQ ID NO:3)

Another recombinant TLAL comprises the C-terminal 317 amino acid residues of hLAL, and in another embodiment, consists of those 317 amino acid residues. The N-terminus of this truncated form of rhLAL corresponds to Phe83 of hLAL. This truncated form is designated TLAL-F83 (SEQ ID NO:4).

Another recombinant TLAL comprises the C-terminal 318 amino acid residues of hLAL, and in another embodiment, consists of those 318 amino acid residues. The N-terminus of this truncated form of rhLAL corresponds to Val82 of hLAL. This truncated form is designated TLAL-V82 (SEQ ID NO:5).

Another recombinant TLAL comprises the C-terminal 319 amino acid residues of rhLAL, and in another embodiment, consists of those 319 amino acid residues. The N-terminus of this truncated form of rhLAL corresponds to Val81 of hLAL. This truncated form is designated TLAL-V81 (SEQ ID NO:6).

Another recombinant TLAL comprises the C-terminal 320 amino acid residues of hLAL, and in another embodiment, consists of those 320 amino acid residues. The N-terminus of this truncated form of rhLAL corresponds to Pro80 of hLAL. This truncated form is designated TLAL-P80 (SEQ ID NO:7).

Another recombinant TLAL comprises the C-terminal 321 amino acid residues of hLAL, and in another embodiment, consists of those 321 amino acid residues. The N-terminus of this truncated form of rhLAL corresponds to Lys79 of hLAL. This truncated form is designated TLAL-K79 (SEQ ID NO:8).

Another recombinant TLAL comprises the C-terminal 322 amino acid residues of hLAL, and in another embodiment, consists of those 322 amino acid residues. The N-terminus of this truncated form of rhLAL corresponds to Pro78 of hLAL. This truncated form is designated TLAL-P78 (SEQ ID NO:9).

The TLAL proteins described herein may contain a truncation at the C-terminus up to 1, 2, 3, 4, 5, 6, 7, 8, 9, 10, 15 or 20 amino acid residues.

### Isolated Mixture Comprising TLAL

The present invention includes a composition of an isolated mixture of recombinant lysosomal acid lipase proteins comprising a TLAL and at least one more forms of recombinant human LAL (rhLAL). For example, the mixture of the LAL proteins can comprise at least one form of TLAL and at least one form of full-length LAL proteins. The mixture of the LAL proteins can be a mixture comprising two or more different forms of TLAL proteins described herein.

Without wishing to be tied with a theory, it is believed that some TLAL can be produced in mammalian cell culture that expresses a full-length rhLAL by a posttranslational modification (i.e., an intracellular protein maturation process). In one embodiment, the TLAL and the full-length hLAL are recombinant proteins produced from mammalian cell culture. The mixture comprising a full-length rhLAL and a TLAL can be obtained from mammalian cell culture expressing a full-length rhLAL which subsequently undergoes intracellular proteolytic cleavage. Accordingly, the present invention contemplates an isolated mixture comprising a full-length hLAL and a TLAL. The mixture can be obtained from conditioned medium of mammalian cell culture expressing the full-length LAL and be subjected to purification steps in which the various forms of LAL proteins can be further isolated and purified. In one particular embodiment, TLAL is purified away from another form of rhLAL protein contained in the mixture, e.g., the full-length rhLAL protein. In yet another embodiment, the full-length LAL protein is purified away from the TLAL in the mixture.

In one embodiment, an isolated mixture can comprise at least one form of TLAL selected from the group consisting of SEQ ID NOs: 2-58 and a full-length rhLAL. In one particular embodiment, the isolated mixture comprises TLAL-D75 (SEQ ID NO:12), TLAL-K76 (SEQ ID NO:13), or TLAL-G77 (SEQ ID NO:14) and any of the full-length rhLAL proteins described herein. Depending on the length of the signal peptide sequence cleaved off from the unprocessed rhLAL protein, the full-length human LAL present in the mixture can be 378 amino acid residues long (i.e., Ser22 through Gln399), 377 amino acid residues long (i.e., Gly23 through Gln399), 376 amino acid residues long (i.e., Gly24 through Gln399), 375 amino acid residues long (i.e., Lys25 through Gln399), 374 amino acid residues long (i.e., Leu26 through Gln399), 373 amino acid residues long (i.e., Thr27 through Gln399) or 372 amino acid residues long (i.e., Ala28 through Gln399).

In another embodiment, the isolated mixture comprising TLAL can contain at least two forms of TLAL. In one particular embodiment embodiment, the isolated mixture comprising TLAL can contain at least two forms of TLAL selected from the group consisting of TLAL-D75 (SEQ ID NO:12), TLAL-K76 (SEQ ID NO:13) and TLAL-G77 (SEQ ID NO:14).

### LAL Fusion Proteins

The invention further includes fusion proteins, comprising an rhLAL (i.e., full-length LAL or TLAL) as a first moiety, linked to a second moiety that does not occur naturally in a human LAL. The second moiety can be an amino acid or polypeptide that are fused at the N-terminus of the rhLAL, the C-terminus of the rhLAL, or internally to any amino acid residue position from Pro31 to Ala90 of rhLAL (SEQ ID NO:1). As contemplated in the invention, the second moiety can be fused recombinantly or chemically (e.g., covalently) via a linker and/or spacer known in the art.

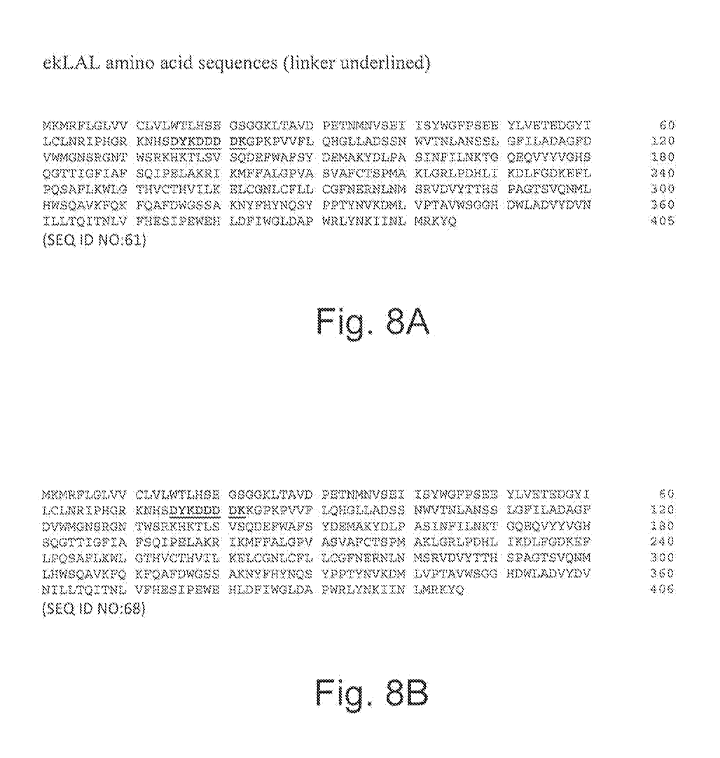

In one embodiment, the fusion protein comprises a TLAL or a full-length rhLAL and a second moiety comprising an affinity ligand, with or without a linker and/or spacer joining the first and the second moieties. The fusion protein comprising an affinity ligand can be produced in a host cell and can be purified from a cell lysate or from the culture medium, using a suitable affinity matrix that binds to the affinity ligand. A non-limiting example of such affinity ligand is the FLAG affinity sequence shown in FIG. 7 (SEQ ID NO:60) which can be used to isolate rhLAL protein containing the ligand sequence (e.g., FIG. 8: SEQ ID NO:61). For use of affinity ligands incorporated into fusion proteins, see Ch. 16, sec. III in Current Protocols in Molecular Biology, Ausubel et al., eds., last updated 11 Jan. 2012.

In one embodiment, the second moiety comprises a proteolytic cleavage sequence with or without a linker and/or spacer joining the first and the second moieties. The proteolytic cleavage sequence can be introduced at any desired position between Pro31 and Ala90 of rhLAL (SEQ ID NO:1), preferably between Pro31 and Gly77 of rhLAL. When a full-length LAL having an internal cleavage site is produced, a proteolytic reaction can be carried out in vitro to allow for cleavage of rhLAL at the desired site. In one specific embodiment, the cleavage sequence is an enterokinase (“ek”) linker (i.e., Asp-Asp-Asp-Asp-Lys; SEQ ID NO:64). Enterokinase is a protease which cleaves the protein after the lysine (K) residue at its cleavage site DDDDK (SEQ ID NO:64).

In some aspects, the fusion protein can be a TLAL protein or a full-length rhLAL containing one or more second moieties that function to target the protein to a specific cell, tissue or organ. In one embodiment, the fusion protein comprises a TLAL and a second moiety comprising a receptor ligand. The targeting moiety is capable of binding to a cell surface receptor present on a target cell, where it is internalized via endocytosis. Useful targeting moieties for directing the TLAL to the target cells include, but are not limited to, p97, insulin-like growth factor (IGF)-I, IGF-II, transferrin receptor ligand, RAP, ApoB, ApoE, aprotinin, lipoprotein lipase, low density lipoprotein receptor-related protein 1 (LRP-1), and variants, homologues or fragments thereof. These targeting moieties can increase rapid internalization, enhance pharmacokinetics, and direct proper targeting of the fusion protein to specific cells, tissues or organs.

In another embodiment, the fusion protein comprises a TLAL or a full-length LAL and a second moiety that functions to modulate serum half-life of the TLAL. For example, polyalkylene oxide conjugates of TLAL or full-length LAL protein are also a part of the invention. In one embodiment, polyethylene glycol (PEG) is conjugated to a TLAL at one or more sites, for example, through one or more epsilon amino groups of lysine residues, via a linker such as succinimidyl carbonate, thiazolidine thione, urethane or amide base linkers. Non-limiting examples of the second moiety that can be fused to the LAL protein by recombinant technology include a Fc region of human immunoglobulin, human serum albumin (HSA), transferrin, β2-macroglobulin, and variants, homologues or fragments thereof.

### Allelic Variants and Modifications

TLAL proteins with amino acid sequences corresponding to those allelic variants of hLAL are also part of the invention. TLAL proteins having an amino acid sequence not corresponding exactly to hLAL, but having one or more (for example, two, three, four or five) conservative amino acid substitutions are also included in the invention. Conservative amino acid substitutions are those substitutions that are made within these designated groups that allow for the retention of the acidic (Asp, Glu, Asn, Gin), basic (His, Lys, Arg), polar (Ser, Cys, Thr, Met), aromatic (Phe, Tyr, Trp) or hydrophobic (Gly, Ala, Val, Leu, lie) properties of the amino acid side chain so long as such substitution allows for enzymatic activity of TLAL.

The present invention also contemplates modified rhLALs and modified TLALs that retain enzymatic activity. In one embodiment, a modified rhLAL comprises the amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98% or 99% identity to the amino acid sequence set forth in SEQ ID NO:1. In another embodiment, a modified TLAL protein comprises the amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98% or 99% identity to any of the amino acid sequences set forth in SEQ ID NOs:2-58.

### Enzymatic Activity

In one embodiment of the invention, the isolated recombinant TLAL has an enzymatic activity of a lysosomal acid lipase (LAL). In another embodiment, the enzymatic activity of a LAL includes, but is not limited to, one or more enzymatic activities selected from the group consisting of hydrolysis of cholesteryl esters and hydrolysis of triglycerides. Assays for determining the enzymatic activity of a LAL are well known to one of ordinary skill in the art. For example, LAL enzymatic activity can be determined by using the fluorogenic substrate 4-methylumbelliferyl-oleate assay as described in Example 12 of WO/2011/133960 and Yan et al. (2006), American Journal of Pathology, 169 (3):916-926, the entire contents of which are incorporated herein by reference. Cellular uptake of an rhLAL can be determined using macrophage and fibroblast cells according to the assay described in Example 13 of WO/2011/133960, the entire contents of which are incorporated herein by reference. Moreover, enzymatic activity of rhLAL can be assessed in vivo in LAL-deficient Yoshida Rats according to the assay described in Example 14 of WO/2011/133960 and Kuriyama et al. (1990), Journal of Lipid Research, vol. 31, p 1605-1611; Nakagawa et al. (1995), Journal of Lipid Research, vol. 36, p 2212-2218; and Yoshida and Kuriyama (1990), Laboratory Animal Science, vol. 40, p 486-489, the entire contents of each of which are incorporated herein by reference,

TLAL proteins of the present invention have an enzymatic level or activity that is at least 50%, 55%, 60%, 65%, 70%, 75%, 80%, 85%, 90%, 95%, 96%, 97%, 98%, 99%, 100%, 105%, 110%, 115%, 120%, 125%, 130%, 135%, 140%, 145%, 150%, 155%, 160%, 165%, 170%, 175%, 180%, 185%, 190%, 195% or even 200% of the normal level of activity observed in a full-length rhLAL. The “normal level” as used herein refers to a level of LAL activity of the full-length hLAL in corresponding wild-type (i.e., LAL⁺/LAL⁺) cells or to a level of LAL activity of an unmodified full-length recombinant human LAL treated with or present in a similar condition.

In some embodiments, the TLAL of the present invention may achieve an enzymatic activity level that is increased by at least 1.25-fold, 1.5-fold, 1.75-fold, or even 2-fold, as compared to positive controls (e.g., a full-length rhLAL treated with or present in a similar condition). In some embodiments, TLAL of the present invention may achieve an enzymatic level or activity at least approximately 10 nmol/hr/mg, 20 nmol/hr/mg, 30 nmol/hr/mg, 40 nmol/hr/mg, 50 nmol/hr/mg, 60 nmol/hr/mg, 70 nmol/hr/mg, 80 nmol/hr/mg, 90 nmol/hr/mg, 100 nmol/hr/mg, 150 nmol/hr/mg, 200 nmol/hr/mg, 250 nmol/hr/mg, 300 nmol/hr/mg, 350 nmol/hr/mg, 400 nmol/hr/mg, 450 nmol/hr/mg, 500 nmol/hr/mg, 550 nmol/hr/mg or 600 nmol/hr/mg in a target tissue.

### Production of TLAL

TLAL can be produced from transgenic animals (e.g., cow, sheep, goat and birds), insect cells, plants, yeast or bacteria as known in the art using the methods described herein. Depending on a host cell and its biological mechanism, the TLAL can be produced directly from an unmodified coding sequence rhLAL or indirectly using proteolytic cleavage which results in deletion of a designed N-terminus region from a longer rhLAL produced.

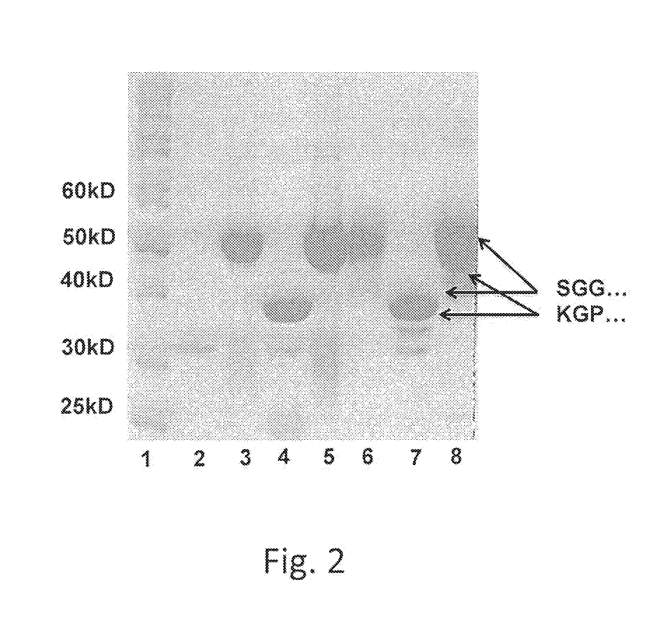

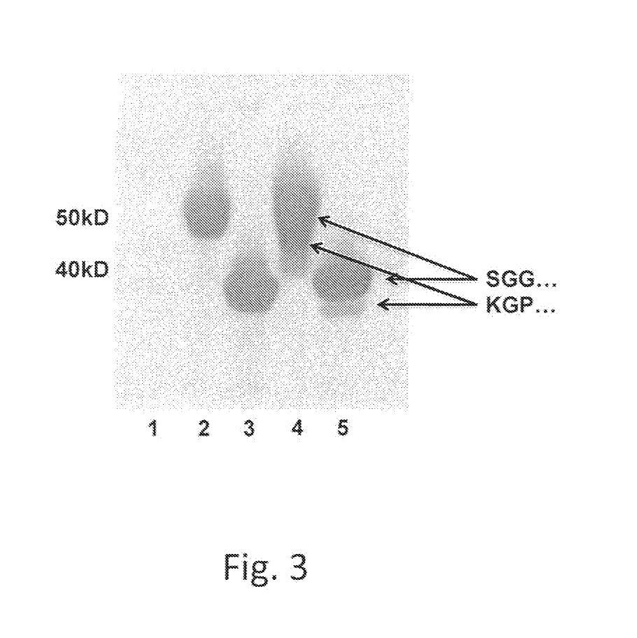

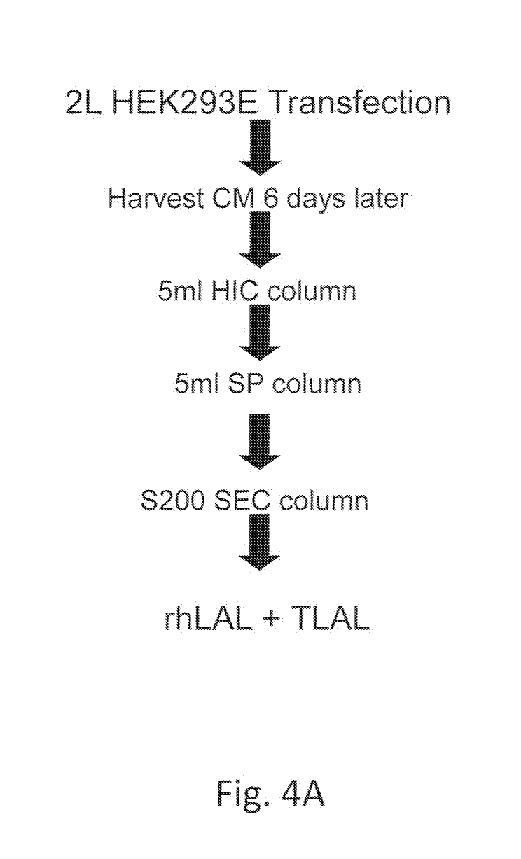

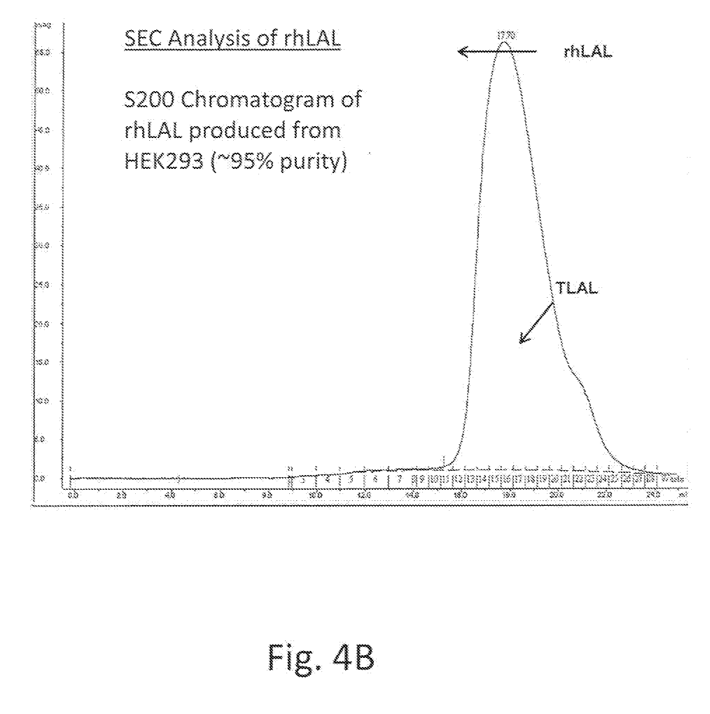

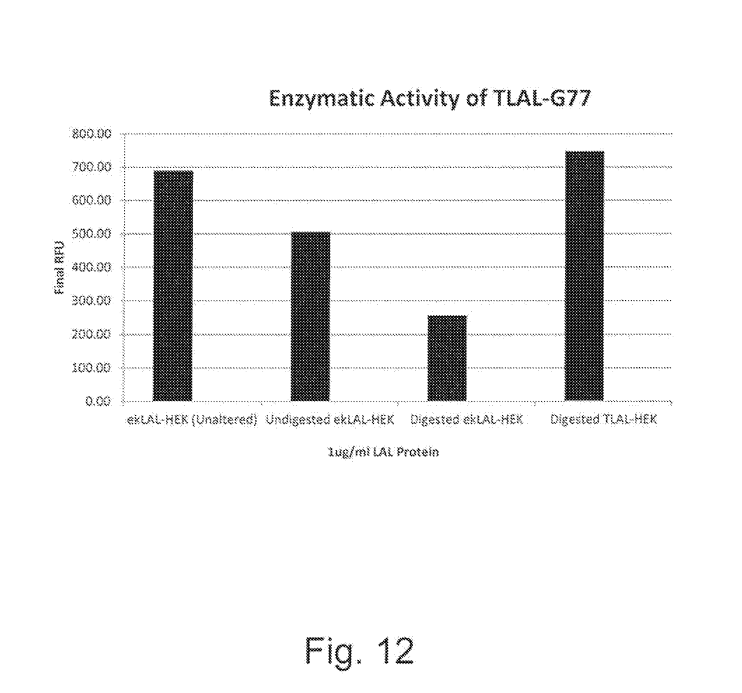

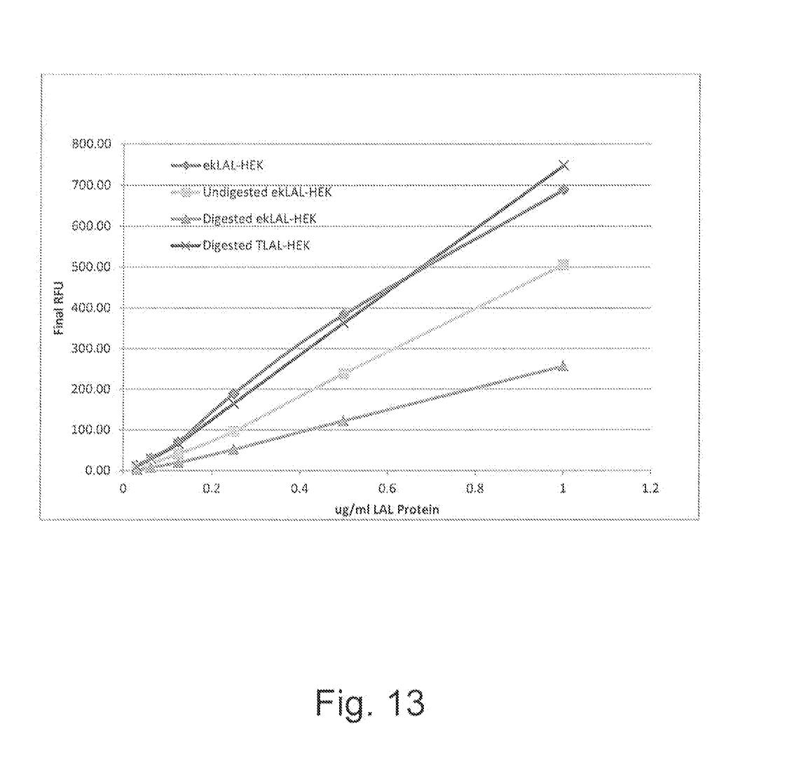

In mammalian cell culture, recombinant human LAL (rhLAL) was observed as a 53 kDa band on SDS-PAGE (“SGP . . . ” in FIGS. 2 and 3) following cleavage of the signal peptide to yield the processed full-length LAL pro-protein. Surprisingly, a lower molecular weight form (apparent 41 kDa band labeled “KGP . . . ”) was found in conditioned media of HEK293 cells expressing full-length rhLAL (FIGS. 2 and 3). Peptide sequencing of this low molecular weight LAL protein revealed that this form of rhLAL was TLAL-K76 (SEQ ID NO:11). Purified TLAL-K76 was determined to have an enzyme activity level that is equal to or higher than that of a full-length rhLAL (Example 4; FIGS. 12 and 13).

Accordingly, the present invention includes recombinant methods to produce TLAL proteins, without requiring introduction of an artificial proteolytic cleavage site. TLAL can be produced from proteolysis of a full length form of hLAL that occurs in a mammalian cell culture (e.g., HEK293 cells). In particular embodiments, the TLAL proteins produced in cultured mammalian cells can be TLAL-K76 (SEQ ID NO:11) and/or TLAL-G77 (SEQ ID NO:10).

Not all TLAL proteins may be stable in all host cells. See, for example, Zschenker et al. (Zschenker, et al., J. Biochem. 136:65-12, 2004) describing the failure to achieve expression of a short form of LAL in Spodoptera frugiperda insect cells. Thus, where TLAL is not adequately expressed in certain host cells, a full-length or any rhLAL having an adequate length of the N-terminal region can be recombinantly modified to introduce an artificial proteolytic cleavage site within the N-terminal region to obtain a TLAL of a desired length.

In one embodiment, a full-length rhLAL comprising a proteolytic cleavage site can be expressed, isolated from host cell culture and subsequently subjected to proteolysis at the cleavage site to obtain the desired TLAL from the full-length rhLAL. A proteolytic cleavage site can be introduced at any desired position between Pro31 and Ala90 of rhLAL (SEQ ID NO:1). When a full-length LAL is produced with an internal cleavage site, a proteolytic reaction can be carried out in vitro to allow for cleavage of the full length proteins.

In one embodiment, the cleavage linker is an enterokinase (“ek”) recognition sequence (i.e., Asp-Asp-Asp-Asp-Lys; SEQ ID NO:64) which is also part of the FLAG affinity sequence. Enterokinase is a specific protease which cleaves the protein after the lysine (K) residue at its cleavage site DDDDK (SEQ ID NO:64).

Alternatively, the proteolytic linker can be the Factor Xa cleavage sequence (i.e., Ile-Glu/Asp-Gly-Arg; SEQ ID NO:65). Factor Xa cleaves a peptide containing the cleavage sequence of Ile-Glu/Asp-Gly-Arg (SEQ ID NO:65) after the arginine residue. A furin cleavage can be also used as a proteolytic linker. As the major processing enzyme of the secretory pathway, furin is a ubiquitous subtilisin-like proprotein convertase having the minimal cleavage site Arg-X-X-Arg′ (SEQ ID NO:66). Genenase I and its cleavage sequence can be also employed to produce TLAL by proteolysis. Genenase I cleaves a protein containing Pro-Gly-Ala-Ala-His-Tyr (SEQ ID NO:67) after the Tyr residue. Other proteases with specific cleavage sites known in the art can be employed without limitation to produce proteolytic cleavage of rhLAL to obtain a desired TLAL.

Suitable mammalian cells that can be used to obtain TLAL include primary cell cultures derived from a mammal at any stage of development or maturity. Mammalian cells also include cells of mammalian origin that have been transformed to divide for an unlimited number of generation, such as human embryonic kidney line (e.g., HEK293), human fibrosarcoma cell line (e.g., HT1080), human cervical carcinoma cells (HeLa), human lung cells (W138), human liver cells (Hep G2), human retinoblasts, BALB/c mouse myeloma line, COS-7, baby hamster kidney cells (e.g., BHK), Chinese hamster ovary cells (e.g., CHO +/−DHFR), mouse Sertoli cells (TM4), rat liver cells (BRL 3A), mouse mammary tumor (e.g., MMT-060562), TRI cells; MRC 5 cells, FS4 cells, monkey kidney cells (e.g., CV1, VERO-76), canine kidney cells (e.g., MDCK). Different host cells can be chosen to ensure its capacity to modify and process a TLAL protein.

The vector encoding the rhLAL protein and/or TLAL can be introduced into host cells for production of the TLAL protein. The host cells can be grown under conditions in which the coding sequence of the TLAL is expressed. The protein can be purified from the culture medium or from cell extracts, as appropriate. In one method, a coding region for a full-length hLAL (comprising cDNA or DNA synthetically produced) can be enzymatically introduced into a vector to operably link the coding region to a promoter and other control regions necessary for gene expression in a suitable protein expression system including, but not limited to, mammalian cells, plant cells, insect cells, yeast, or bacteria. For example, vectors available for use in mammalian host cells include any vector known in the art for expressing a protein in its corresponding expression system, e.g., pTT22 for HEK293 cells and SV40 for CV1 line. Methods for vector construction are generally known to those of ordinary skill in the art. (See, for example, Durocher et al., U.S. Publication 2011/0039339A1).

A variety of expression vector/host systems can be utilized to express TLAL. The microorganisms include bacteria transformed with recombinant bacteriophage, plasmid, or cosmid DNA expression vectors; yeast transformed with yeast expression vectors, insect cells or plant cells infected with virus expression vectors (e.g., baculovirus). Furthermore, the present invention also contemplates TLAL proteins produced in any useful transgenic expression system including, without limitation, transgenic mammals, and in plant systems including tobacco plant and duck weed.

The control elements or regulatory sequences include those non-translated regions of the vector, enhancers, promoters, 5′ and 3′ untranslated regions which interact with host cellular proteins to carry out transcription and translation. Such elements can vary in their strength and specificity. Depending on the vector system and host utilized, any number of suitable transcription and translation elements, including, tissue-specific, constitutive and inducible promoters, can be used. In mammalian cell systems, promoters from mammalian genes or from mammalian viruses are preferred. If a cell line that contains multiple copies of the sequence encoding TLAL is desired, vectors based on SV40 or EBV can be used with an appropriate selectable marker.

The present invention also contemplates production of TLAL in a transgenic avian system. When an avian expression system is used, the vectors described in U.S. Pat. No. 6,730,822; U.S. Pat. No. 6,825,396; U.S. Pat. No. 6,875,588; U.S. Pat. No. 7,294,507; U.S. Pat. No. 7,521,591; U.S. Pat. No. 7,534,929; and U.S. Patent Publication No. 2006/0185024 are preferred. The present invention includes production of TLAL with or without a second moiety (e.g., targeting or proteolytic cleavage sequence). The avian expression vector can include one or more regulatory sequences such as oviduct-specific promoter, for example, and without limitation, ovomucoid promoters, ovalbumin promoters, lysozyme promoters, conalbumin promoters, ovomucin promoters, ovotransferrin promoters and functional portions of each of these promoters. Suitable non-specific promoters can include, for example and without limitation, cytomegalovirus (CMV) promoters, MDOT promoters and Rous-sarcoma virus (RSV) promoters, murine leukemia virus (MLV) promoters, mouse mammary tumor virus (MMTV) promoters and SV40 promoters and functional portions of each of these promoters. Non-limiting examples of other promoters which can be useful in the present invention include, without limitation, Pol III promoters (for example, type 1, type 2 and type 3 Pol III promoters) such as HI promoters, U6 promoters, tRNA promoters, RNase MPR promoters and functional portions of each of these promoters. Typically, functional terminator sequences are selected for use in accordance with the promoter that is employed.

TLAL can be glycosylated on one, two, three, four, five or six Asn (N) sites for N-linked glycosylation depending on the length of the TLAL and the expression system in which it is produced. The full-length LAL has six potential glycosylation sites at Asn36, Asn72, Asn101, Asn161, Asn273 and Asn321. The invention includes all forms of glycosylated TLAL proteins, glycosylated at any or all of these sites. The present invention also contemplates an unglycosylated TLAL protein. In some embodiments, Asn72 may not be glycosylated (see for example, PCT/US2011/033699; WO/2011/133960). In one embodiment, the TLAL protein can comprise one or more glycan structures at the C-terminal Asn101, Asn161, Asn273 and Asn321 glycosylation sites. In another embodiment, the TLAL protein may have one or more glycan structures at the Asn36, Asn101, Asn161, Asn273 and Asn321 glycosylation sites. In yet another embodiment, the TLAL protein may contain one or more glycan structures at the Asn72, Asn101, Asn161, Asn273 and Asn321 glycosylation sites. In still another embodiment, the TLAL protein may contain one or more glycan structures at Asn36, Asn72, Asn101, Asn161, Asn273 and Asn321 glycosylation sites.

### Purification of TLAL

A TLAL can be purified to various grades of purity. The purity of TLAL in a composition is at least equal to or greater than 80%, 82%, 85%, 87%, 90%, 93%, 95%, 96%, 97%, 98%, or 99% by total protein as determined by HPLC methods. The composition can be free of any detectable band of about 30 kDa to 37 kDa on SDS-PAGE. The composition can be free of any detectable band of about 50 kDa to 60 kDa on SDS-PAGE. A composition comprising TLAL can be purified to a grade that is essentially free of non-LAL protein. A composition comprising TLAL can also be purified to a state wherein the TLAL protein is essentially pure. A TLAL can be present in a solution or in a lyophilized preparation. The composition can be also present in varying proportions of TLAL to a full-length form (rhLAL), such as 10% to 20%, 20% to 30%, 30% to 40%, 40% to 50%, 50% to 60%, 60% to 70%, 70% to 80%, 80% to 90% and 90% to 100%.

### Pharmaceutical Composition

Pharmaceutical compositions comprising one or more forms of TLAL are contemplated in the invention. In some embodiments, the compositions comprising truncated LAL also comprise an aqueous or physiologically compatible fluid suspension or solution. Compositions of TLAL can comprise any nontoxic substance compatible with enzymatic activity and stability. Carrier substances can include sterile water, buffers such as sodium phosphate, potassium phosphate, and other physiologically acceptable buffers such as citrate, bicarbonate and acetate buffers, salts such as sodium chloride and potassium chloride, carbohydrates such as mannitol, maltose, sucrose, sorbitol, xylitol and glucose, alcohol, glycerol, amino acids, urea, EDTA, EGTA, polyvinylpyrollidone, polysaccharides, methylcellulose, sodium carboxymethyl cellulose, propylene glycol, polyethylene glycol, glycerol, ascorbic acid, lipids, phospholipids, buffering agents, dispersing agents, surfactants such as poloxamer 188, polysorbate 80, Cremophore-EL, Cremophore-R, labrofil, and the like. Compositions comprising TLAL can also comprise other proteins, such as full-length rhLAL, impurities not fully purified from the TLAL, or added proteins such as human serum albumin, collagen and gelatin. In some embodiments, the composition can contain benzyl alcohol, phenol or m-cresol. Compositions comprising any combination of the above are included in the invention.

In a specific example, recombinant human TLAL produced as disclosed herein, is employed in a pharmaceutical formulation wherein each 1 milliliter contains TLAL (e.g., 2 mg LAL), trisodium citrate dehydrate (e.g., 13.7 mg), citric acid monohydrate (e.g., 1.57 mg), and human serum albumin (e.g., 10 mg), and is formulated to an acidic pH such as 5.9±0.1.

Compositions comprising LAL can be lyophilized or stored as liquids. For preservation of activity in the lyophilized form, bulking agents such as glycine, mannitol, albumin and dextran can be useful. For cryoprotection, disaccharides, amino acids and polyethylene glycol can be useful. When sugars are included in the composition, the sugar can be included in the concentration of about 2% to about 40% weight to volume. In some embodiments, the composition comprising truncated LAL has a pH in the range of 4.0 to 6.0.

A TLAL or a composition comprising a TLAL can be used alone or in combination with other pharmaceutical agents or therapies in methods to treat a human with a deficiency of hLAL. Methods of administering a TLAL, dosing regimens, methods for evaluating the effectiveness of treatments, and pharmaceutical compositions and formulations for TLAL are as described for recombinant human LAL in U.S. patent application Ser. No. 13/229,558, filed Sep. 9, 2011, which is hereby incorporated by reference in its entirety.

Suitable formulations for administration may contain a pharmaceutical composition at various concentrations. In some embodiments, suitable formulations may contain a TLAL at a concentration up to about 500 mg/mL, e.g., up to about 400 mg/mL, up to about 300 mg/mL, about 250 mg/mL, up to about 200 mg/mL, up to about 150 mg/mL, up to about 100 mg/mL, up to about 90 mg/mL, up to about 80 mg/mL, up to about 70 mg/mL, up to about 60 mg/mL, up to about 50 mg/mL, up to about 40 mg/mL, up to about 30 mg/mL, up to about 25 mg/mL, up to about 20 mg/mL, up to about 10 mg/mL or up to about 5 mg/mL. In some embodiments, suitable formulations may contain a TLAL at a concentration ranging between about 1-500 mg/mL (e.g., about 1-250 mg/mL, about 1-200 mg/mL, about 1-150 mg/mL, about 1-100 mg/mL, about 10-100 mg/mL, about 1-80 mg/mL, about 1-70 mg/mL, about 1-60 mg/mL, about 1-50 mg/mL, about 1-40 mg/mL, about 1-30 mg/mL).

The present invention contemplates any route of administration which facilitates the uptake of TLAL into the lysosomes of pertinent cells, organs and tissues. In some embodiments, formulations suitable for intravenous administration can contain TLAL at a concentration of about 1 mg/mL, about 2 mg/mL, about 3 mg/mL, about 4 mg/mL, about 5 mg/mL, about 10 mg/mL, about 15 mg/mL, about 20 mg/mL, about 25 mg/mL, about 50 mg/mL, about 75 mg/mL, about 100 mg/mL, about 150 mg/mL, about 200 mg/mL, about 250 mg/mL or 300 mg/ml.

### Treatment of LAL Deficiency

The recombinant LAL proteins described herein that exhibit enzyme activity levels at least 50% of the full-length can be employed to treat LAL deficiency (e.g., Wolman disease and cholesteryl ester storage disease). The invention provides methods of treating human patients suffering from LAL deficiency comprising administering TLAL or LAL fusion protein to the patient, wherein the administration is sufficient to restore growth, to improve liver function, to reduce liver damage, to increase tissue levels of LAL, and/or increase LAL activity in the patient. For the treatment of LAL deficiency, generally, the amount of TLAL or LAL fusion protein administered can vary depending on known factors such as age, health, and weight of the recipient, type of concurrent treatment, frequency of treatment, and the like. Usually a dosage of active ingredient can be between about 0.01 and about 50 mg per kilogram of body weight. In one embodiment, dosage of LAL in accordance with the invention is between about 0.1 and 0.5 mg per kilogram of body weight. In one embodiment, the dose is between about 0.1 mg and about 5.0 mg per kilogram. In one embodiment, the dose is about 0.1 mg to about 5.0 mg per kilogram. In one embodiment, the dose is about 0.1 mg to about 10 mg per kilogram. In one embodiment, the dose is about 0.1 mg to about 20 mg per kilogram. In one embodiment the dose is about 0.1, about 0.2, about 0.25, about 0.30, about 0.35, about 0.40, about 0.45, about 0.50 mg per kilogram body weight. In one embodiment, the dose is between about 1 mg and about 5 mg per kilogram body weight. In one embodiment, the dose is about 1 mg per kilogram body weight. In one embodiment, the dose is about 3 mg per kilogram body weight. For example, 0.1 mg per kilogram of body weight, 0.2 mg per kilogram of body weight, 0.3 mg per kilogram of body weight, 0.4 mg per kilogram of body weight, 0.5 mg per kilogram of body weight, 1 mg per kilogram of body weight, 2 mg per kilogram of body weight, 3 mg per kilogram of body weight, 4 mg per kilogram of body weight, or 5 mg per kilogram of body weight can be administered.

In some aspects, the recombinant LAL proteins described herein can be administered to a human patient once in about 4, 5, 6, 7, 8, 9, 10, 11, 12, 13, 14, 15, 16, 17, 18, 19, 20,21,22, 23, 24, 25 or 30 days at a dose about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0 or 10 mg per kilogram of body weight. In one embodiment, the LAL protein is administered weekly, biweekly, or monthly.

In some aspects, the patient is subject to an escalating dosage regimen. For example, the patient can be administered initially with a dose, e.g., about 0.1 to about 0.5 mg per kilogram, for about two weeks or about one month and later with a high dose above 0.5 mg per kilogram of body weight. In some other aspects, the patient can be administered initially with a high dose and later with a dose lower than the initial dose during the treatment.

The therapeutic proteins can be injected by, for example, subcutaneous injections, intramuscular injections, and intravenous (IV) infusions or injections. In one embodiment, the TLAL or the LAL fusion protein is administered intravenously by IV infusion by any useful method. In one example, the TLAL or the LAL fusion protein can be administered by intravenous infusion through a peripheral line. In another example, the TLAL or the LAL fusion protein can be administered by intravenous infusion through a peripherally inserted central catheter. In another example, the TLAL or the LAL fusion protein can be administered by intravenous infusion facilitated by an ambulatory infusion machine attached to a venous vascular access port. In one embodiment, of intravenous infusion, the medication is administered over a period of 1 to 8 hours depending on the amount of medication to be infused and the patient's previous infusion-related reaction history, as determined by a physician skilled in the art. In another embodiment, the TLAL or the LAL fusion protein is administered intravenously by IV injection. In another embodiment, the TLAL or the LAL fusion protein can be administered via intraperitoneal injection. In still another embodiment, the TLAL is administered via a pharmaceutically acceptable capsule of the therapeutic protein. For example, the capsule can be an enteric-coated gelatin capsule.

In some embodiments, the therapeutic proteins are administered by infusion, and the infusion can occur over an extended time period, for example, 30 minutes to 10 hours. Thus, the infusion can occur, for example, over a period of about 1 hour, about 2 hours, about 3 hours, about 4 hours, or about 5 hours. The infusion can also occur at various rates. Thus, for example, the infusion rate can be between about 1 mL per hour and 20 mL per hour. In some embodiments, the infusion rate is between 5 mL and 10 mL per hour. In one embodiment, the infusion rate is 1, 2, 3, 4, 5, 6, 7, 8, 9, 10, 11, 12, 13, 14, 15, 16, 17, 18, 19 or 20 mL per hour. In one embodiment, the infusion rate is between 0.1 and 5 mg/kg/hr. In one embodiment, the infusion rate is about 0.1, about 0.2, about 0.3, about 0.5, about 1.0, about 1.5, about 2.0, or about 3 mg/kg/hr.

Various methods of administering a TLAL protein, dosing regimens, methods for evaluating the effectiveness using a recombinant TLAL are as described for recombinant human LAL in U.S. patent application Ser. No. 13/229,558, filed Sep. 9, 2011, which is hereby incorporated by reference in its entirety.

### EXAMPLES

### Example 1

### Identification and Isolation of TLAL-K76 and TLAL-G77

HEK293E cells (human embryonic kidney cell line stably expressing EBNA-1 protein of Epstein-Barr virus) were transfected with pTT22-LAL (pTT22 with insertion of native human LAL coding region (SEQ ID NO:1); see Publication No. U.S. 2011/0039339 for pTT22 vector) using polyethylenimine. The culture medium was supplemented with peptone TNI 24-48 hours post transfection. The culture remained at a cell density of about 2×10⁶ cells/mL. Medium was collected 6 days after transfection. Protein was purified through a phenyl hydrophobic interaction (HIC) column as well as SP Sepharose™ column, and followed by purification on a S200 size exclusion chromatography (SEC) column.

Analysis of purified samples by SDS-polyacrylamide gel electrophoresis (SDS-PAGE) on a 12% polyacrylamide gel stained with Coomassie blue showed the presence of two forms of rhLAL co-purified (FIG. 2). The apparent molecular weight (MW) of the full-length rhLAL (“SGG . . . ”) was 53 kDa and 43 kDa following digestion with peptide N-glycosidase F-amidase (PNGase F) and that of the smaller form of rhLAL (“KGP . . . ”) was approximately 41-43 kDa (FIG. 2).

To determine whether the smaller protein was derived from rhLAL, proteins were analyzed on Western blot. The proteins were transferred from the gel to a polyvinylidene fluoride (PVDF) membrane using a Pierce Fast Semi-Dry Blotter. The primary and secondary antibodies were rabbit α-LIPA pAb (Abnova catalog no. H00003988-R01) and goat α-rabbit-HRP IgG (H+L) pAb (Thermo Scientific Pierce catalog no. 31460), respectively. ECL Western Blotting Substrate (Thermo Scientific Pierce) was used on the membrane to allow for detection of chemiluminescence in an Alpha Innotech Gel Imaging Cabinet (Scion Corporation) (FIG. 3).

Subsequently, the smaller LAL protein was purified from conditioned media containing both the long and the small form of rhLAL. N-terminal amino acid sequencing revealed that the larger rhLAL protein (53 kD) was indeed a full-length rhLAL having the N-terminal amino acid sequence of SGG and that the smaller form of rhLAL (41-43 kD) was a truncated LAL having the N-terminal amino acid sequence of KGP. The truncated rhLAL was named TLAL-K76, and confirmed to have the amino acid sequence of positions 76-399 of SEQ ID NO:1 (i.e., SEQ ID NO:11; see, FIG. 1).

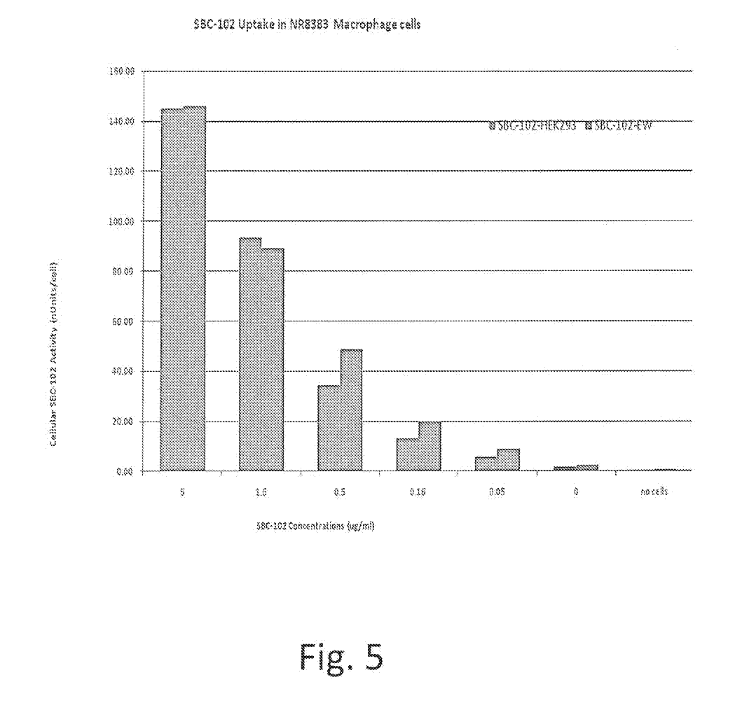

To determine whether the full-length rhLAL produced in HEK293 cells is capable of being internalized into target cells and enzymatically active, human macrophage (NR8383) cells were co-incubated with various concentration of the rhLAL-HEK (“SBC-102-HEK293”). rhLAL produced from transgenic chicken (“SBC-102-EW”) were used as positive control. Subcellular enzyme activity of LAL was measured (units/cell) as described in PCT/US2011/033699 (WO2011/133960). As shown in FIG. 5, the full-length rhLAL produced in HEK293 cell showed similar enzymatic activity levels as compared to rhLAL produced in transgenic chicken.

### Example 2

### Production of EK-LAL

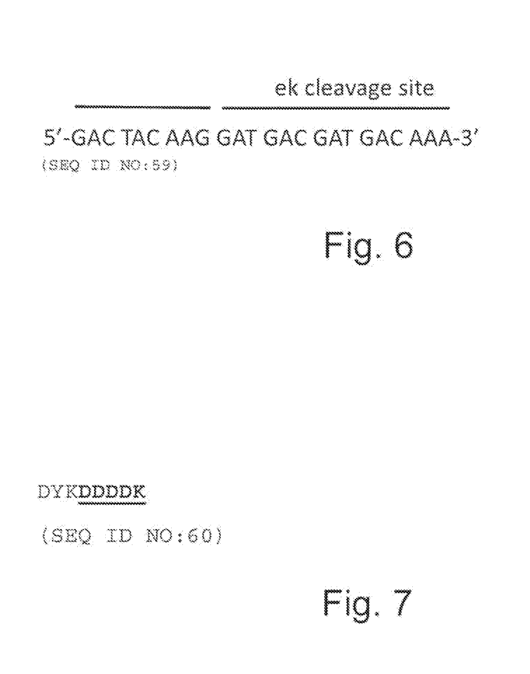

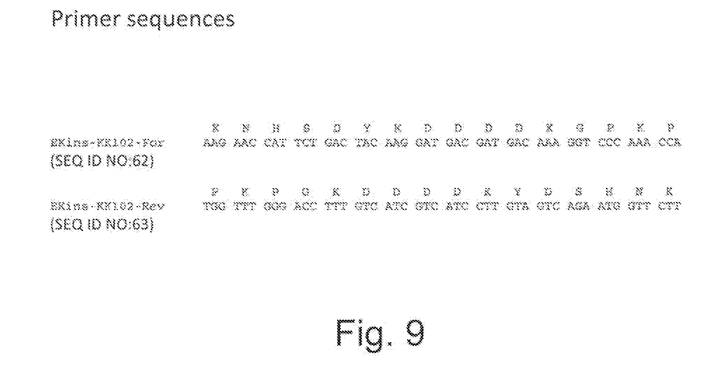

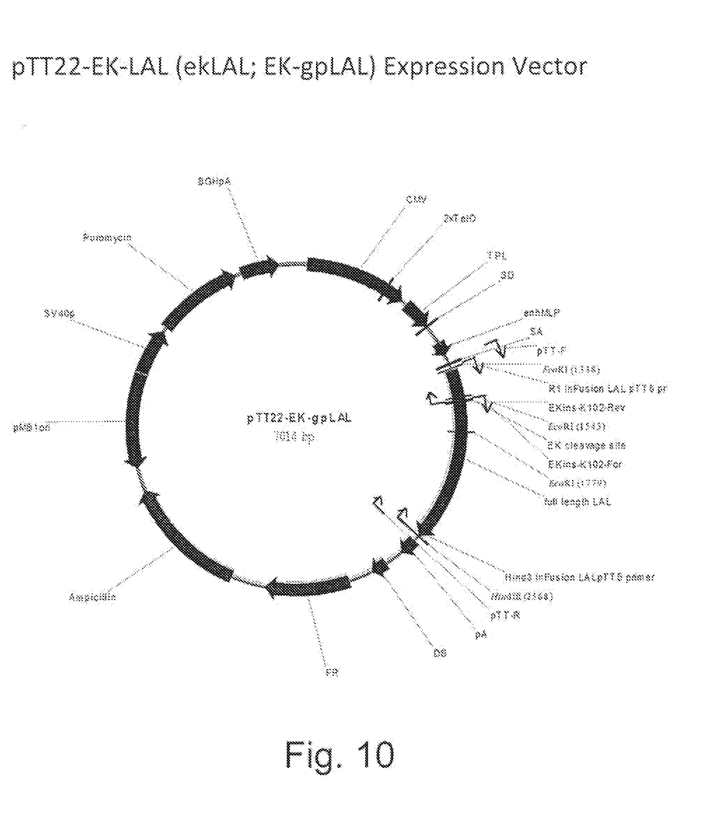

The vector pTT22-EK-gp-LAL was constructed starting from pTT22-LAL. Forward and reverse primers (FIG. 9; SEQ ID NOs: 62 and 63) having the sequence coding the enterokinase (EK) cleavage site as shown in FIG. 6 were used with KOD (from Thermococcus kodakaraensis) polymerase to amplify the entire pTT22-LAL plasmid as template and generate a plasmid with the insertion of nucleotides encoding DYKDDD between S74 and D75 of hLAL (as shown in SEQ ID NO:1). The extra amino acid residues DYK were included as part of an affinity recognition site by the anti-FLAG affinity column. With D and K residues at positions 75 and 76 naturally occurring in hLAL, the insertion resulted in a DDDDK (see FIGS. 7 and 8) recognition sequence for ek (bovine enterokinase; EC 3.4.21.9), which cleaves C-terminal to K, leaving G77 as the N-terminal amino acid residue of a truncated protein having the amino acid sequence of positions 77-399 of SEQ ID NO:1 (also see, SEQ ID NO:10). The resulting plasmid encoding an rhLAL fusion protein (with the insertion of the ek cleavage site) was designated pTT22-EK-gpLAL. A map of pTT22-EK-gpLAL is shown in FIG. 10 and the corresponding amino acid sequence of ekLAL produced from expression of pTT22-EK-gpLAL is shown in FIG. 8. Another plasmid having an insertion of the nucleotide sequence encoding DYKDDDDK (SEQ ID N0:60) was also prepared to create the . . . S-DYKDDDDK-K . . . cleavage sequence in a similar manner. This construct was designed to create TLAL having K76 as the N-terminal amino acid residue after a digestion with ek (i.e., TLAL having the amino acid sequence 76-399 of SEQ ID NO:1; also see, SEQ ID NO:11).

HEK293 cells were transfected with the plasmid described above and grown in cell culture. Culture medium from ekLAL-HEK293 cells was adjusted to 20 mM Na3P04, 137 mM NaCl, pH 6.0, final volume of 2 liters. The hydrophobic interaction column (HIC) (HiTrap Phenyl Sepharose 6 Fast Flow™, GE Healthcare™, Cat. No. 17-5193-01) base on a 90 μm matrix was loaded with samples and washed with 20 mM NasPCU, 137 mM NaCl, pH 6.0, then washed with 5 mM Na₃PO₄ pH 6.0. Protein was eluted with 5 mM Tris, 50% propylene glycol, pH 7.2. Fractions containing protein with maximal enzymatic activity were used for further purification on an ion exchange (IEX) column (HiTrap SP Fast Flow™, GE Healthcare, Cat. No. 17-5157-01). SP Sepharose™ Fast Flow is a cation exchanger based on a 6% highly cross-linked beaded agarose matrix. The IEX column was loaded with the pooled fractions adjusted to 50 mM NaOAc, pH 5.0 and washed with buffer of the same salts. Proteins were eluted with 50 mM NaOAc, 35 mM CaCl2, pH 5.0. Pooled fractions from the IEX column were used for further purification on an α-FLAG affinity column (Anti-FLAG M2™ Affinity Gel, Sigma, Cat. No. A2220) equilibrated with TBS, pH 7.4, with elution using 1M Tris, pH 8.0. The affinity resin in the α-FLAG affinity column bound to the octapeptide DYKDDDDK (SEQ ID NO:60) of ekLAL. Fractions containing maximal activity of ekLAL were eluted, pooled and concentrated.

Enzymatic activity of ekLALs containing the octapeptide DYKDDDDK (SEQ ID NO:60) tag in the N-terminal region was measured as described the following section. Contrary to the findings by Zschenker et al. (2004) that the N-terminal region of LAL is essential for protein folding, stability, secretion and enzyme activity, ekLAL proteins containing the octapeptide in the N-terminal region (e.g., SEQ ID NOs:61 and 64) exhibited enzyme activity levels similar to that of the unmodified full-length wild-type rhLAL, suggesting that the mutations created by insertion of the octapeptide in the N-terminal region did not affect the enzyme activity of rhLAL.

### Example 3

### Enterokinase (ek) Digestion and Production of TLAL-K76 and TLAL-G77

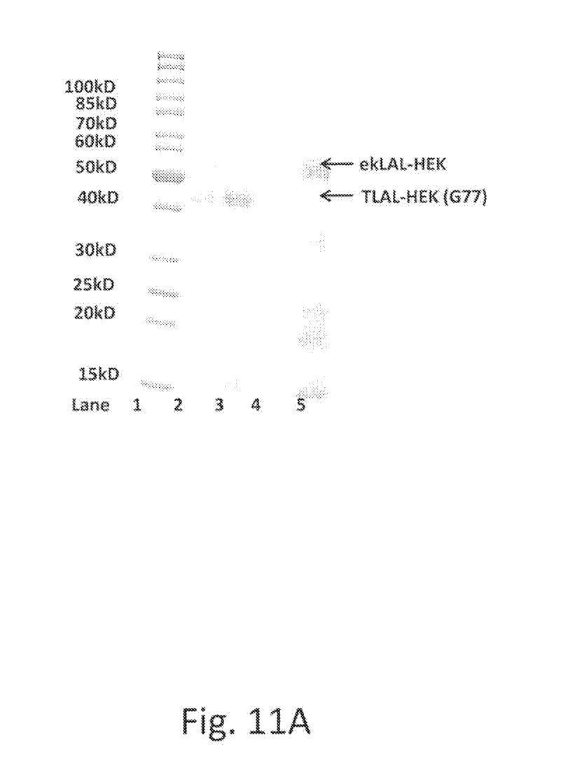

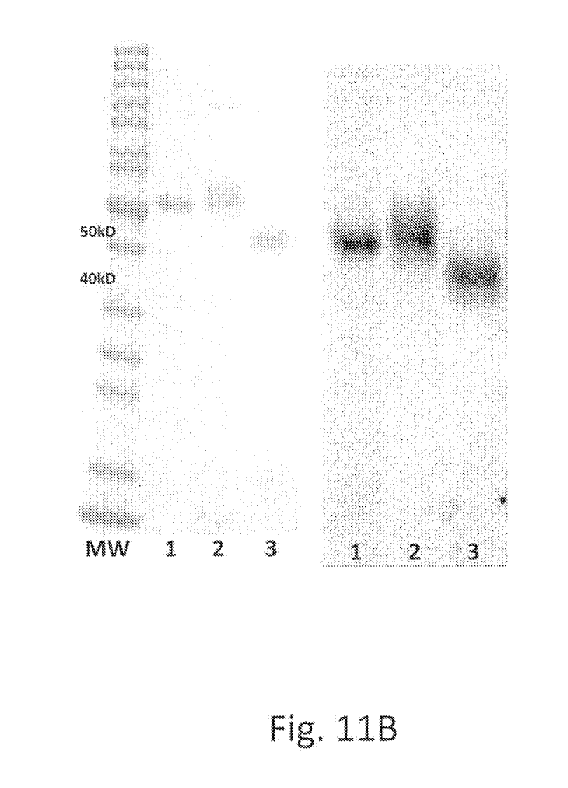

Purified samples of ek tagged LAL (i.e., SEQ ID NO: 61 and 68) were added to rEK reaction buffer (5 μL of 10× EK buffer in 45 μL of dH₂O). Enterokinase (1:20 units to protein ratio) was added to initiate digestion reactions, and the reaction pool was incubated at room temperature for 6 days. After digestion reaction, proteins were purified by the α-FLAG affinity column to separate the TLAL-HEK, from ekLAL-HEK (full-length). Various α-FLAG affinity column fractions were run on 12% SDS-PAGE (see, e.g., FIG. 11A for TLAL-G77). TLAL was purified essentially free of any protein between ˜30 kDa and ˜37 kDa and shown to be approximately 40-43 kDa (see, FIG. 11A, lane 3 for TLAL-G77; and FIG. 11B, lane 3 for TLAL-K76). The ek digestion of ekLAL of SEQ ID NO:61 resulted in approximately 50% cleavage into TLAL-G77 (see, FIG. 11A, lane 2) whereas the ek digestion of ekLAL of SEQ ID NO:64 resulted >70% cleavage into TLAL-K76 (data not shown). rhLAL protein produced from transgenic chicken (“LAL-EW”), HEK293 (“LAL-HEK”) and unaltered (i.e., assayed immediately after thawing) ekLAL-HEK were used as controls to ensure that the LAL protein was not randomly degraded after being incubated for 6 days at room temperature for digestion. Molecular weight of undigested LAL-EW and LAL-HEK and those of digested appeared to be the same, indicating that non-specific ek cleavage did not occur during the digestion reaction.

### Example 4

### In Vitro Analysis of TLAL Activity

Enzyme activity of various forms of rhLAL including full-length rhLAL, rhLAL proteins containing the ek cleavage site (ekLALs), and TLAL was determined using the fluorogenic substrate 4-methylumbelliferyl-oleate assay essentially as described in Yan et al. (2006), American Journal of Pathology, Vol. 169, No. 3, p 916-926, the disclosure of which is incorporated herein by reference in its entirety.

A substrate stock solution of 2.5 mM 4-methylumbelliferyl oleate (4-MUO) in 4% Triton X-100 was prepared. The assay was performed in a microtiter plate, each well containing 62.5 μL of 0.2 M sodium citrate (pH 5.5) in 0.01% Tween80, 12.5 μL of LAL samples and 25 μL of the 2.5 mM 4-MUO. Change in fluorescence was monitored for 30 minutes at 37° C. using a Bio-Tek Synergy HT fluorometric microplate reader (excitation 360 nm and emission 460 nm). Prior to assay, samples containing rhLAL was diluted to a concentration that resulted in the reaction continuing linearly for at least 30 minutes. The reaction was stopped with 50 μL of 0.75 M Tris-HCl, pH 8.0 and the endpoint fluorescence signal was measured in the same plate reader used above (excitation 360 nm and emission 460 nm). Units of activity were determined using 4-methylumbelliferyl as a standard. One unit (U) is defined as the amount of enzyme which results in the formation of 1 μmole of 4-methylumbelliferyl/min under the assay conditions described above.

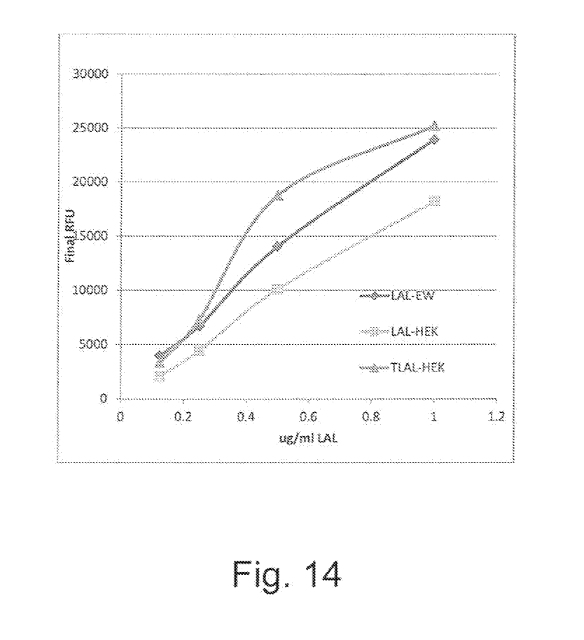

TLALs exhibited enzymatic activity levels that are similar to or higher than that of similarly treated ekLALs (see, FIGS. 12 and 13) or that of unmodified wild-type full-length rhLAL (FIG. 14).

Each example in the above specification is provided by way of explanation of the invention, not limitation of the invention. In fact, it will be apparent to those skilled in the art that various modifications, combinations, additions, deletions and variations can be made in the present invention without departing from the scope or spirit of the invention. For instance, features illustrated or described as part of one embodiment can be used in another embodiment to yield a still further embodiment. It is intended that the present invention cover such modifications, combinations, additions, deletions, and variations.

All publications, patents, patent applications, internet sites, and accession numbers/database sequences (including both polynucleotide and polypeptide sequences) cited herein are hereby incorporated by reference in their entirety for all purposes to the same extent as if each individual publication, patent, patent application, internet site, or accession number/database sequence were specifically and individually indicated to be so incorporated by reference.

1-30. (canceled)

31. A fusion protein comprising a recombinant human lysosomal acid lipase (rhLAL) or a truncated human lysosomal acid lipase (TLAL) and a second moiety,
wherein said second moiety is an affinity ligand, a targeting moiety, or a moiety that modulates serum half-life.

32. The fusion protein of claim 31, wherein the fusion protein comprises TLAL and the TLAL is selected from the group consisting of SEQ IDS NOs: 11-58.

33. The fusion protein of claim 32, wherein the TLAL comprises the amino acid sequence set forth in SEQ ID NO: 11.

34. The fusion protein of claim 31, wherein the second moiety is fused to the N-terminus of the rhLAL or TLAL, the C-terminus of the rhLAL or TLAL, or internally to any amino acid residue position between Pro31 and Gly77 of the rhLAL or TLAL.

33. The fusion protein of claim 31, wherein the second moiety is fused to the rhLAL or TLAL via a linker.

36. The fusion protein of claim 31, wherein the second moiety is an affinity ligand.

37. The fusion protein of claim 36, wherein the affinity ligand comprises a FLAG affinity sequence.

38. The fusion protein of claim 31, wherein the second moiety is a targeting moiety.

39. The fusion protein of claim 38, wherein the targeting moiety selected from a group consisting of p97, insulin-like growth factor (IGF)-I, IGF-11 transferrin receptor ligand, RAP, ApoB, ApoE, aprotinin, lipoprotein lipase, low density lipoprotein receptor-related protein 1 (LRP-1), and variants, homologies or fragments thereof.

40. The fusion protein of claim 31, wherein the second moiety is a moiety that modulates serum half-life.

41. The fusion protein of claim 40, wherein the moiety that modulates serum half-life is selected from a group consisting of polyalkylene oxide conjugates human serum albumin (HSA), transferrin, α2-macroglobulin, and variants homologues or fragments thereof.

42. A pharmaceutical composition comprising the fusion protein of claim 31.

43. A method for treating a Lysosomal Acid Lipase (LAL) deficiency in a patient, the method comprising:
administering to the patient a pharmaceutical composition of claim 42 in an amount effective to treat LAL deficiency.

44. The method of claim 43, wherein the LAL deficiency is Wolman disease (WD).

45. The method of claim 43, wherein the LAL deficiency is cholesteryl ester storage disease (CESD).

46. The method of claim 43, wherein the pharmaceutical composition is administered intravenously.

47. The method of claim 43, wherein, the pharmaceutical composition is administered via a pump.

48. The method of claim 43, wherein the amount is in range of 0.1 to 20 mg of fusion protein per kg of body weight.


SEQUENCE LISTING (68 sequences)

SEQ ID NO: 1 - protein, 399 aa, Homo sapiens
MKMRFLGLVVCLVLWTLHSEGSGGKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 2 - protein, 315 aa, Homo sapiens
QHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 3 - protein, 316 aa, Homo sapiens
LQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 4 - protein, 317 aa, Homo sapiens
FLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 5 - protein, 318 aa, Homo sapiens
VFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 6 - protein, 319 aa, Homo sapiens
VVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 7 - protein, 320 aa, Homo sapiens
PVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 8 - protein, 321 aa, Homo sapiens
KPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 9 - protein, 322 aa, Homo sapiens
PKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 10 - protein, 323 aa, Homo sapiens
GPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 11 - protein, 324 aa, Homo sapiens
KGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 12 - protein, 325 aa, Homo sapiens
DKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 13 - protein, 326 aa, Homo sapiens
SDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 14 - protein, 327 aa, Homo sapiens
HSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 15 - protein, 328 aa, Homo sapiens
NHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 16 - protein, 329 aa, Homo sapiens
KNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 17 - protein, 330 aa, Homo sapiens
RKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 18 - protein, 331 aa, Homo sapiens
GRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 19 - protein, 332 aa, Homo sapiens
HGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 20 - protein, 333 aa, Homo sapiens
PHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 21 - protein, 334 aa, Homo sapiens
IPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 22 - protein, 335 aa, Homo sapiens
RIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 23 - protein, 336 aa, Homo sapiens
NRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 24 - protein, 337 aa, Homo sapiens
LNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 25 - protein, 338 aa, Homo sapiens
CLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 26 - protein, 339 aa, Homo sapiens
LCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 27 - protein, 340 aa, Homo sapiens
ILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 28 - protein, 341 aa, Homo sapiens
YILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 29 - protein, 342 aa, Homo sapiens
GYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 30 - protein, 343 aa, Homo sapiens
DGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 31 - protein, 344 aa, Homo sapiens
EDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 32 - protein, 345 aa, Homo sapiens
TEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 33 - protein, 346 aa, Homo sapiens
ETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 34 - protein, 347 aa, Homo sapiens
VETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 35 - protein, 348 aa, Homo sapiens
LVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 36 - protein, 349 aa, Homo sapiens
YLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 37 - protein, 350 aa, Homo sapiens
EYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 38 - protein, 351 aa, Homo sapiens
EEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 39 - protein, 352 aa, Homo sapiens
SEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 40 - protein, 353 aa, Homo sapiens
PSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 41 - protein, 354 aa, Homo sapiens
FPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 42 - protein, 355 aa, Homo sapiens
GFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 43 - protein, 356 aa, Homo sapiens
WGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 44 - protein, 357 aa, Homo sapiens
YWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 45 - protein, 358 aa, Homo sapiens
SYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 46 - protein, 359 aa, Homo sapiens
ISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 47 - protein, 360 aa, Homo sapiens
IISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 48 - protein, 361 aa, Homo sapiens
EIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 49 - protein, 362 aa, Homo sapiens
SEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 50 - protein, 363 aa, Homo sapiens
VSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 51 - protein, 364 aa, Homo sapiens
NVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 52 - protein, 365 aa, Homo sapiens
MNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 53 - protein, 366 aa, Homo sapiens
NMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 54 - protein, 367 aa, Homo sapiens
TNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 55 - protein, 368 aa, Homo sapiens
ETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 56 - protein, 369 aa, Homo sapiens
PETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 57 - protein, 370 aa, Homo sapiens
DPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 58 - protein, 371 aa, Homo sapiens
VDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 59 - DNA, 24 bp, Homo sapiens
gactacaaggatgacgatgacaaa

SEQ ID NO: 60 - protein, 8 aa, Homo sapiens
DYKDDDDK

SEQ ID NO: 61 - protein, 405 aa, Homo sapiens
MKMRFLGLVVCLVLWTLHSEGSGGKLTAVDPETNMNVSEIISYWGFPSEEYLVETEDGYILCLNRIPHGRKNHSDYKDDDDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 62 - DNA, 48 bp, Homo sapiens
aagaaccattctgactacaaggatgacgatgacaaaggtcccaaacca

SEQ ID NO: 63 - DNA, 48 bp, Homo sapiens
tggtttgggacctttgtcatcgtcatccttgtagtcagaatggttctt

SEQ ID NO: 64 - protein, 5 aa, Artificial Sequence
  porcine
DDDDK

SEQ ID NO: 65 - protein, 4 aa, Artificial Sequence
  Homo sapiens
  misc_feature (2)..(2): Xaa can be Glu or Asp
IXGR

SEQ ID NO: 66 - protein, 4 aa, Artificial Sequence
  Homo sapiens
  misc_feature (2)..(3): Xaa can be any naturally occurring amino acid
RXXR

SEQ ID NO: 67 - protein, 6 aa, Artificial Sequence
  Bacillus subtilis
PGAAHY

SEQ ID NO: 68 - protein, 406 aa, Homo sapiens
MKMRFLGLVVCLVLWTLHSEGSGGKLTAVDPETNMNVSEIISYWGFPSEEYLVETEDGYILCLNRIPHGRKNHSDYKDDDDKKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ